SEQUENCE LISTING (4 sequences)

SEQ ID NO: 1 - DNA, 19 bp, Homo sapiens
gctttcaggatagtcgtga

SEQ ID NO: 2 - DNA, 19 bp, Homo Sapiens
agcgagacttcatggacga

SEQ ID NO: 3 - DNA, 19 bp, Homo Sapiens
tggtggtatacaatgcagg

SEQ ID NO: 4 - DNA, 19 bp, Homo Sapiens
gtctaccagatcttcgata

(19) United States
(12) Patent Application Publication
(10) Pub. No.: US 2011/0059526 A1
(43) Pub. Date: Mar. 10, 2011

(54) REPROGRAMMING A CELL BY INDUCING A PLURIPOTENT GENE THROUGH USE OF AN HDAC MODULATOR

(75) Inventors: Kenneth J. Eilertsen, Baton Rouge, LA (US); Rachel A. Power, Zachary, LA (US); Jong S. Rim, Baton Rouge, LA (US)

(73) Assignee: NUPOTENTIAL, INC., Baton Rouge, LA (US)

(21) Appl. No.: 12/616,983

(22) Filed: Nov. 12, 2009

Related U.S. Application Data
(60) Provisional application No. 61/113,971, filed on Nov. 12, 2008.

(51) Int. Cl. C12N 5/02; C12N 5/00

(52) U.S. Cl. 435/377; 435/325

(57) ABSTRACT

The invention relate to methods, compositions, and kits for reprogramming a cell. In one embodiment, the invention relates to a method comprising inducing the expression of at least one gene that contributes to a cell being pluripotent or multipotent. In yet another embodiment, the method comprises inhibiting the activity of an HDAC with an HDAC inhibitor and inducing the expression of at least one gene that contributes to a cell being pluripotent or multipotent. In still another embodiment, the invention relates to a method for reprogramming comprising exposing a cell to more than one agent to inhibit more than ore type of regulatory protein. In yet another embodiment, the invention relates to a reprogrammed cell or an enriched population of reprogrammed cells that can have characteristics of an ES-like cell, which can be re- or trans-differentiated into various differentiated cell types

### CROSS REFERENCE TO RELATED PATENT APPLICATIONS

This application claims benefit under 35 U.S.C. §119(e) of U.S. Provisional Application 61/113,971, filed Nov. 12, 2008, which is incorporated herein by reference as if set forth in its entirety.

### FIELD

Embodiments of the invention relate to the fields of cell biology, stem cells, cell differentiation, somatic cell nuclear transfer and cell-based therapeutics. More specifically, embodiments of the invention are related to methods, compositions and kits for reprogramming cells and cell-based therapeutics.

### BACKGROUND

Regenerative medicine holds great promise as a therapy for many human ailments, but also entails some difficult technical challenges, which include low cloning efficiency, a short supply of potentially pluripotent tissues, and a generalized lack of knowledge as to how to control cell differentiation and what types of embryonic stem cells can be used for selected therapies. While ES cells have tremendous plasticity, undifferentiated ES cells can form teratomas (benign tumors) containing a mixture of tissue types. In addition, transplantation of ES cells from one source to another likely would require the administration of drugs to prevent rejection of the new cells.

Attempts have been made to identify new avenues for generating stem cells from tissues that are not of fetal origin. One approach involves the manipulation of autologous adult stem cells. The advantage of using autologous adult stem cells for regenerative medicine lies in the fact that they are derived from and returned to the same patient, and are therefore not subject to immune-mediated rejection. A drawback is that these cells lack the plasticity and pluripotency of ES cells and thus their potential is uncertain. Another approach is aimed at reprogramming somatic cells from adult tissues to create pluripotent ES-like cells. However, this approach has been difficult as each cell type within a multi-cellular organism has a unique epigenetic signature that is thought to become fixed once cells differentiate or exit from the cell cycle.

Cellular DNA generally exists in the form of chromatin, a complex comprising of nucleic acid and protein. Indeed, most cellular RNA molecules also exist in the form of nucleoprotein complexes. The nucleoprotein structure of chromatin has been the subject of extensive research, as is known to those of skill in the art. In general, chromosomal DNA is packaged into nucleosomes. A nucleosome comprises a core and a linker. The nucleosome core comprises an octamer of core histones (two each of H2A, H2B, H3 and H4) around which is wrapped approximately 150 base pairs of chromosomal DNA. In addition, a linker DNA segment of approximately 50 base pairs is associated with linker histone H1. Nucleosomes are organized into a higher-order chromatin fiber and chromatin fibers are organized into chromosomes. See, for example, Wolffe “Chromatin: Structure and Function” 3.sup.rd Ed., Academic Press, San Diego, 1998.

Chromatin structure is not static, but is subject to modification by processes collectively known as chromatin remodeling. Chromatin remodeling can serve, for example, to remove nucleosomes from a region of DNA; to move nucleosomes from one region of DNA to another; to change the spacing between nucleosomes; or to add nucleosomes to a region of DNA in the chromosome. Chromatin remodeling can also result in changes in higher order structure, thereby influencing the balance between transcriptionally active chromatin (open chromatin or euchromatin) and transcriptionally inactive chromatin (closed chromatin or heterochromatin).

Chromosomal proteins are subject to numerous types of chemical modification. One mechanism for the posttranslational modification of these core histones is the reversible acetylation of the epsilon-amino groups of conserved highly basic N-terminal lysine residues. The steady state of histone acetylation is established by the dynamic equilibrium between competing histone acetyltransferase(s) and histone deacetylase(s) herein referred to as HDAC.

HDACs are classified in at least four classes depending on sequence identity and domain organization: Class I: HDAC1, HDAC2, HDAC3, HDAC8; Class II: HDAC4, HDAC5, HDAC6, HDAC7A, HDAC9, HDAC10; Class III: sirtuins in mammals (SIRT1, SIRT2, SIRT3, SIRT4, SIRT5, SIRT6, SIRT7); and Class IV: HDAC11. Class I HDACs are those that most closely resemble the yeast transcriptional regulator RPD3. Class II HDACs are those that most closely resemble the yeast HDA1 enzyme.

Histone acetylation and deacetylation has long been linked to transcriptional control. The reversible acetylation of histones can result in chromatin remodeling and as such can act as a control mechanism for gene transcription. In general, hyperacetylation of histones facilitates gene expression, whereas histone deacetylation is correlated with transcriptional repression. Histone acetyltransferases were shown to act as transcriptional coactivators, whereas deacetylases were found to belong to transcriptional repression pathways.

The dynamic equilibrium between histone acetylation and deacetylation is essential for normal cell growth. Inhibition of histone deacetylation results in cell cycle arrest, cellular differentiation, apoptosis and reversal of the transformed phenotype.

The development of pluripotent or totipotent cells into a differentiated, specialized phenotype is determined by the particular set of genes expressed during development. Gene expression is mediated directly by sequence-specific binding of gene regulatory proteins that can effect either positive or negative regulation. However, the ability of any of these regulatory proteins to directly mediate gene expression depends, at least in part, on the accessibility of their binding site within the cellular DNA. As discussed above, accessibility of sequences in cellular DNA often depends on the structure of cellular chromatin within which cellular DNA is packaged.

Therefore, it would be useful to identify methods, compositions and kits that can induce the expression of genes required for pluripotency, including methods, compositions, and kits that can inhibit the activity of HDACs involved in repressing transcription.

### BRIEF SUMMARY

The invention relates to methods, compositions and kits for reprogramming a cell. Embodiments of the invention relate to methods comprising inducing the expression of a pluripotent or multipotent gene. In yet another embodiment, the invention further relates to producing a reprogrammed cell. In still yet another embodiment, the invention relates to a method comprising inhibiting the activity, expression or activity and expression of at least one HDAC by use of an HDAC inhibitor. In yet another embodiment, the invention relates to a method comprising altering the activity, expression or activity and expression of at least one HDAC by use of an HDAC modulator. The method further comprises inducing the expression of at least one pluripotent or multipotent gene, and reprogramming the cell.

Embodiments of the invention also relate to methods for reprogramming a cell comprising contacting a cell, a population of cells, a cell culture, a subset of cells from a cell culture, a homogeneous cell culture or a heterogeneous cell culture with an HDAC modulator, inducing the expression of at least one pluripotent or multipotent gene, and reprogramming the cell. The method further comprises re-differentiating the reprogrammed cell.

In another embodiment, the invention relates to the use of an agent to inhibit the expression, activity or expression and activity of an HDAC. The agent can be any molecule or compound that can inhibit the expression, activity, or expression and activity of an HDAC including but not limited to an HDAC inhibitor, a small molecule, a nucleic acid sequence, a DNA sequence, an RNA sequence, a shRNA sequence, and RNA interference.

In another embodiment, the invention relates to the use of an agent to induce the activity, expression, or activity and expression of a protein that inhibits the activity of an HDAC. The agent can be any molecule or compound that can induce the expression, activity, or expression and activity of a protein that inhibits an HDAC including but not limited to a small molecule, a nucleic acid sequence, a DNA sequence, an RNA sequence, a shRNA sequence, and RNA interference

An HDAC inhibitor can be used to inhibit the activity of an HDAC and includes but is not limited to TSA, sodium butyrate, valproic acid, vorinostat, LBH-589, apicidin, TPX-HA analogue, CI-994, MS-275, MGCD0103, and derivatives or analogues of the above-mentioned.

In some embodiments, at least one HDAC inhibitor can inhibit at least one HDAC. In still yet another embodiment, more than one HDAC inhibitor, either simultaneously or sequentially, can inhibit at least one HDAC. An HDAC inhibitor can be directed toward an HDAC in class I, class II, class III, class IV, or an unknown or unclassified HDAC. An HDAC inhibitor can be directed toward more than one class of HDACs or all classes of HDACs. Combinations of HDAC inhibitors can inhibit more than one HDAC, and can be used simultaneously or sequentially.

In another embodiment, the invention relates to a method for reprogramming a cell comprising: exposing a population of cells to an agent that inhibits activity, expression, or activity and expression of a histone deacetylase; inducing expression of a pluripotent or multipotent gene; selecting a cell that express a cell surface marker indicative of a pluripotent or multipotent cell, and expanding said selected cell to produce a population of cells, wherein differentiation potential has been restored to said cell.

In yet another embodiment, the invention relates to a method for reprogramming a cell comprising: exposing a cell to a first agent that inhibits that activity, expression or expression and activity of a HDAC; exposing said cell to a second agent that inhibits the activity, expression or expression and activity of a second regulatory protein, wherein said second regulatory protein has a distinct function from the HDAC, inducing expression of a pluripotent or multipotent gene, and selecting a cell, wherein differentiation potential has been restored to said cell. In another embodiment, the cell or population of cells may be exposed to the first and second agent simultaneously or sequentially.

In still another embodiment, the invention relates to a method for reprogramming a cell comprising: exposing a population of cells to an agent that inhibits activity, expression, or activity and expression of a HDAC, wherein the inhibition of at least one HDAC leads to an increase in expression of other HDACs; exposing said population of cells to a second agent that inhibits activity, expression, or activity and expression of the other HDACs; selecting a cell that expresses a cell surface marker indicative of a pluripotent cell, and expanding said selected cell to produce a population of cells, wherein differentiation potential has been restored to said cell.

In still another embodiment, the invention relates to a method for reprogramming a cell comprising: exposing a cell to a first agent that inhibits that activity, expression, or expression and activity of a HDAC; wherein the inhibition of at least one HDAC leads to an increase in expression of a second regulatory protein; exposing said cell to a second agent that inhibits the activity, expression or expression and activity of a second regulatory protein, wherein said second regulatory protein has a distinct function from the HDAC, and selecting a cell, wherein differentiation potential has been restored to said cell.

In still another embodiment, the invention relates to a method comprises exposing a cell with a first phenotype to an agent that inhibits the activity, expression or activity and expression of at least one HDAC; comparing the first phenotype of the cell to a phenotype obtained after exposing the cell to said agent, and selecting the cell that has been reprogrammed. In yet another embodiment, the method comprises comparing the genotype of a cell prior to exposing the cell to said agent to a genotype of the cell obtained after exposing said cell to said agent. In still yet another embodiment, the method comprises comparing the phenotype and genotype of a cell prior to exposing the cell to an agent that inhibits the activity, expression or activity and expression of at least one HDAC to the phenotype and genotype of the cell after exposing the cell to said agent.

In still another embodiment, the method comprises culturing or expanding the selected cell to a population of cells. In yet another embodiment, the method comprises isolating a cell using an antibody that binds to a protein coded for by a pluripotent or multipotent gene or an antibody that binds to a multipotent marker or a pluripotent marker, including but not limited to SSEA3, SSEA4, Tra-1-60, and Tra-1-81. Cells may also be isolated using any method efficient for isolating cells including but not limited to a fluorescent cell activated sorter, immunohistochemistry, and ELISA. In another embodiment, the method comprises selecting a cell that has a less differentiated state than the original cell.

In still another embodiment, the invention further comprises comparing chromatin structure of a pluripotent or multipotent gene prior to exposure to said agent to the chromatin structure obtained after exposure to said agent.

In another embodiment, the invention relates to a method for reprogramming a cell comprising: exposing a cell with a first transcriptional pattern to an agent that inhibits the activity, expression or activity and expression of a HDAC; inducing expression of a pluripotent or multipotent gene; comparing the first transcriptional pattern of the cell to a transcriptional pattern obtained after exposure to said agent; and selecting a cell, wherein differentiation potential has been restored to said cell.

In still another embodiment, selecting a cell comprises identifying a cell with a transcriptional pattern that is at least 1-5%, 5-10%, 10-20%, 20-30%, 30-40%, 40-50%, 50-60%, 60-70%, 70-80%, 80-90%, 90-94%, 95%, or 95-99% similar to a transcriptional pattern of an embryonic stem cell. The entire transcriptional pattern of an embryonic stem cell need not be compared, although it may. A subset of embryonic genes may be compared including but not limited to 1-5, 5-10, 10-25, 25-50, 50-100, 100-200, 200-500, 500-1,000, 1,000-2,000, 2,000-2,500, 2,500-5,000, 5,000-10,000 and greater than 10,000 genes. The transcriptional patterns may be compared in a binary fashion, i.e., the comparison is made to determine if the gene is transcribed or not. In another embodiment, the rate and/or extent of transcription for each gene or a subset of genes may be compared. Transcriptional patterns can be determined using any methods known in the art including but not limited to RT-PCR, quantitative PCR, a microarray, southern blot and hybridization.

Embodiments of the invention also include methods comprising treating a variety of diseases using a reprogrammed cell produced according to the methods disclosed herein. In yet another embodiment, the invention also relates to therapeutic uses for reprogrammed cells and reprogrammed cells that have been re-differentiated.

Embodiments of the invention also relate to a reprogrammed cell produced by the methods of the invention. The reprogrammed cell can be re-differentiated into a single lineage or more than one lineage. The reprogrammed cell can be multipotent or pluripotent.

In yet another embodiment, the invention relates to an enriched population of reprogrammed cells produced according to a method comprising the steps of: exposing a population of cells to an agent that inhibits activity, expression of activity and expression of a histone deacetylase; inducing expression of a pluripotent or multipotent gene; selecting a cell that express a cell surface marker indicative of a pluripotent or multipotent cell, and expanding said selected cell to produce a population of cells, wherein differentiation potential has been restored to said cell.

In another embodiment, the invention relates to an enriched population of reprogrammed cells produced according to a method comprising: exposing a population of cells to an agent that inhibits activity, expression, or activity and expression of a histone deacetylase, wherein said inhibition of at least one HDAC leads to an increase in expression of a second regulatory protein; exposing said cell to a second agent that inhibits the activity, expression or expression and activity of said second regulatory protein; and selecting a cell that express a cell surface marker indicative of a pluripotent cell. The second regulatory protein can be any protein involved in transcriptional regulation, translation, DNA binding, DNA melting, maintaining the cellular state of a cell including but not limited to a histone acetyltransferase, a lysine methyltransferase, a histone methyltransferase, a histone demethylase, a lysine demethylase, a sirtuin, and a sirtuin activator.

The first agent, the second agent, and the first and second agent can be any compound, molecule or nucleic acid construct that achieves the desired effect including but not limited to a small molecule inhibitor, a nucleic acid sequence, and a shRNA construct.

In still another embodiment, the reprogrammed cell expresses a cell surface marker indicative of a pluripotent cell selected from the group consisting of: SSEA3, SSEA4, Tra-1-60, and Tra-1-81. In still another embodiment, the reprogrammed cell expresses a pluripotent gene including but not limited to Oct-4, Sox-2 and Nanog. In yet another embodiment, the reprogrammed cells account for at least 1-5%, 5-10%, 10-20%, 20-30%, 30-40%, 40-50%, 50-60%, 60-70%, 70-80%, 80-90%, 90-95%, 96-98%; or at least 99% of the enriched population of cells.

Embodiments of the invention also relate to kits for preparing the methods and compositions of the invention. The kit can be used for, among other things, reprogramming a cell and generating ES-like and stem cell-like cells.

### BRIEF DESCRIPTION OF THE DRAWINGS

FIG. 1 is a bar graph reporting the up-regulation of Oct-4 in primary human lung cells treated with valproic acid (VPA).

FIG. 2 is a bar graph reporting the up-regulation of several genes, which confer stem-cell like characteristics, in primary human lung cells treated with an HDAC inhibitor (VPA).

FIG. 3 is an illustration reporting the demethylation of two cytosines in the first exon of Oct-4 in cells treated with VPA.

FIG. 4A is a graph reporting the effects on the gene Nanog as measured by fold-change in mRNA expression during HDAC7 or HDAC11 shRNA interference in adult human dermal fibroblasts. FIG. 4B is a graph reporting the effects on the gene Nanog as measured by fold-change in mRNA expression during HDAC7 or HDAC11 shRNA interference in neonatal human dermal fibroblasts. FIG. 4C is a graph reporting the effects on the gene Nanog as measured by fold-change in mRNA expression during HDAC7 or HDAC11 shRNA interference in fetal human dermal fibroblasts.

FIG. 5A is a graph reporting the effects on the gene Oct-4 as measured by fold-change in mRNA expression during HDAC7 or HDAC11 shRNA interference in adult human dermal fibroblasts. FIG. 5B is a graph reporting the effects on the gene Oct-4 as measured by fold-change in mRNA expression during HDAC7 or HDAC11 shRNA interference in neonatal human dermal fibroblasts. FIG. 5C is a graph reporting the effects on the gene Oct-4 as measured by fold-change in mRNA expression during HDAC7 or HDAC11 shRNA interference in fetal human dermal fibroblasts.

FIG. 6 is a graph reporting the effects on the gene Sox-2 as measured by fold-change in mRNA expression during HDAC7 or HDAC11 shRNA interference in fetal human dermal fibroblasts.

FIG. 7 is a graph reporting the effects on various HDAC and SIRT genes as measured by mRNA expression during HDAC7 shRNA interference in human dermal fibroblasts.

FIG. 8 is a graph reporting the effects on the gene Nanog as measured by fold-change in mRNA expression during dual HDAC7 and HDAC11 shRNA interference in adult human dermal fibroblasts (HDFa), neonatal human dermal fibroblasts (HDFn), and fetal human dermal fibroblasts (HDFf).

FIG. 9 is a graph reporting the effects on the gene Oct-4 as measured by fold-change in mRNA expression during dual HDAC7 and HDAC11 shRNA interference in adult human dermal fibroblasts (HDFa), neonatal human dermal fibroblasts (HDFn), and fetal human dermal fibroblasts (HDFf).

FIG. 10 is a graph reporting the effects on the gene Sox-2 as measured by fold-change in mRNA expression during dual HDAC7 and HDAC11 shRNA interference in adult human dermal fibroblasts (HDFa), neonatal human dermal fibroblasts (HDFn), and fetal human dermal fibroblasts (HDFf).

FIG. 11 is a graph reporting the effects on various HDAC genes and SIRT genes as measured by fold change in mRNA expression during dual HDAC7 and HDAC11 shRNA interference in adult human dermal fibroblasts.

FIG. 12 is a graph reporting the effects on various HDAC genes and SIRT genes as measured by fold change in mRNA expression during dual HDAC7 and HDAC11 shRNA interference in fetal human dermal fibroblasts.

FIG. 13 is a graph reporting the effects on various HDAC genes and SIRT genes as measured by fold change in mRNA expression during dual HDAC7 and HDAC11 shRNA interference in neonatal human dermal fibroblasts.

FIG. 14A is a graph reporting the effect of HDAC7a shRNA on the expression of HDAC7a and HDAC11 in adult human dermal fibroblasts. Data for cells grown both in the absence and presence of puromycin are reported.

FIG. 14B is a graph reporting the effect of HDAC7a shRNA on the expression of HDAC7a and HDAC11 in neonatal human dermal fibroblasts. Data for cells grown both in the absence and presence of puromycin are reported.

FIG. 14C is a graph reporting the effect of HDAC7a shRNA on the expression of HDAC7a and HDAC11 in fetal human dermal fibroblasts.

FIG. 15A is a photograph of fetal human dermal fibroblasts.

FIG. 15B is a photograph of fetal human dermal fibroblasts infected with DNMT1 shRNA.

FIG. 15C is a photograph of fetal human dermal fibroblasts infected with HDAC7 shRNA.

FIG. 15D is a photograph of fetal human dermal fibroblasts infected with DNMT1 and HDAC7 shRNA.

FIG. 15E is a photograph of fetal human dermal fibroblasts infected with DNMT1 and HDAC11 shRNA.

FIG. 15F is a photograph of fetal human dermal fibroblasts infected with HDAC11 and HDAC7 shRNA.

FIG. 15G is a photograph of human embryonic stem cells.

FIG. 16A is a photograph of fetal human dermal fibroblasts.

FIG. 16B is a photograph of fetal human dermal fibroblasts infected with DNMT1 shRNA.

FIG. 16C is a photograph of fetal human dermal fibroblasts infected with DNMT1 and HDAC7 shRNA.

FIG. 16D is a photograph of fetal human dermal fibroblasts infected with DNMT1 and HDAC11 shRNA.

FIG. 16E is a photograph of fetal human dermal fibroblasts infected with HDAC7 shRNA.

FIG. 16F is a photograph of fetal human dermal fibroblasts infected with HDAC11 and HDAC7 shRNA.

FIG. 16G is a photograph of human embryonic stem cells.

FIG. 17 is a bar graph reporting the effects of VPA (5 mM) on gene expression of Oct-4, Sox-2 and Nanog in human dermal fibroblasts. Significance relative to control within each cell type: *p<0.05; **p<0.03; and ***p<0.01.

FIG. 18 is a bar graph reporting the effects of VPA (500 μM) on HDAC and DNMT1 gene expression in BJ dermal fibroblasts (obtained from ATCC).

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

“Cell” or “cells,” unless specifically limited to the contrary, includes any somatic cell, embryonic stem (ES) cell, adult stem cell, an organ specific stem cell, nuclear transfer (NT) units, and stem-like cells. The cell or cells can be obtained from any organ or tissue. The cell or cells can be human or other animal. For example, a cell can be mouse, guinea pig, rat, cattle, horses, pigs, sheep, goats, etc. A cell also can be from non-human primates.

“Culture Medium” or “Growth Medium” means a suitable medium capable of supporting growth of cells.

“Differentiation” means the process by which cells become structurally and functionally specialized during embryonic development.

“Epigenetics” means the state of DNA with respect to heritable changes in function without a change in the nucleotide sequence. Epigenetic changes can be caused by modification of the DNA, such as by methylation and demethylation, without any change in the nucleotide sequence of the DNA.

“Histone” means a class of protein molecules found in chromosomes responsible for compacting DNA enough so that it will fit within a nucleus.

“Histone deacetylase inhibitor” and “inhibitor of histone deacetylase” mean a compound that is capable of interacting with a histone deacetylase and inhibiting its enzymatic activity. “Inhibiting histone deacetylase activity” means reducing the ability of a histone deacetylase to remove an acetyl group from a suitable substrate, such as a histone, or other protein. In some embodiments, such reduction of histone deacetylase activity is at least about 10-25%, in other embodiments at least about 50%, in other embodiments at least about 75%, and still in other embodiments at least about 90%. In still yet other embodiments, histone deacetylase activity is reduced by at least 95% and in other embodiments by at least 99%.

“Knock down” means to suppress the expression of a gene in a gene-specific fashion. A cell that has one or more genes “knocked down,” is referred to as a knock-down organism or simply a “knock-down.”

“Pluripotent” means capable of differentiating into cell types of the 3 germ layers or primary tissue types.

“Pluripotent gene” means a gene that contributes to a cell being pluripotent.

“Pluripotent cell cultures” are said to be “substantially undifferentiated” when that display morphology that clearly distinguishes them from differentiated cells of embryo or adult origin. Pluripotent cells typically have high nuclear/cytoplasmic ratios, prominent nucleoli, and compact colony formation with poorly discernable cell junctions, and are easily recognized by those skilled in the art. It is recognized that colonies of undifferentiated cells can be surrounded by neighboring cells that are differentiated. Nevertheless, the substantially undifferentiated colony will persist when cultured under appropriate conditions, and undifferentiated cells constitute a prominent proportion of cells growing upon splitting of the cultured cells. Useful cell populations described in this disclosure contain any proportion of substantially undifferentiated pluripotent cells having these criteria. Substantially undifferentiated cell cultures may contain at least about 20%, 40%, 60%, or even 80% undifferentiated pluripotent cells (in percentage of total cells in the population).

“Regulatory protein” means any protein that regulates a biological process, including regulation in a positive and negative direction. The regulatory protein can have direct or indirect effects on the biological process, and can either exert affects directly or through participation in a complex.

“Reprogramming” means removing epigenetic marks in the nucleus, followed by establishment of a different set of epigenetic marks. During development of multicellular organisms, different cells and tissues acquire different programs of gene expression. These distinct gene expression patterns appear to be substantially regulated by epigenetic modifications such as DNA methylation, histone modifications and other chromatin binding proteins. Thus each cell type within a multicellular organism has a unique epigenetic signature that is conventionally thought to become “fixed” and immutable once the cells differentiate or exit the cell cycle. However, some cells undergo major epigenetic “reprogramming” during normal development or certain disease situations.

“Totipotent” means capable of developing into a complete embryo or organ.

Embodiments of the invention relate to methods comprising inducing the expression of at least one gene that contributes to a cell being pluripotent or multipotent. In another embodiment, the invention relates to methods comprising inducing the expression of at least one gene that contributes to a cell being multipotent. In some embodiments, the methods comprise inducing expression of at least one gene that contributes to a cell being pluripotent or multipotent and producing reprogrammed cells that are capable of directed differentiation into at least one lineage.

Embodiments of the invention also relate to methods comprising modifying chromatin structure, and reprogramming a cell to be pluripotent or multipotent. In yet another embodiment, modifying chromatin structure comprises inhibiting the activity of an HDAC.

In another embodiment, the method comprises inhibiting the activity of an HDAC, and inducing expression of at least one gene that contributes to a cell being pluripotent or multipotent. In yet another embodiment, the method comprises inhibiting the activity of an HDAC and producing a reprogrammed cell.

In still another embodiment, the invention relates to a method for reprogramming a cell comprising: exposing a cell to an agent that inhibits the activity, expression or activity and expression of an HDAC, inducing expression of a pluripotent or multipotent gene; and selecting a cell, wherein differentiation potential has been restored to said cell. The pluripotent or multipotent gene may be induced by any fold increase in expression including but not limited to 0.25-0.5, 0.5-1, 1.0-2.5, 2.5-5, 5-10, 10-15, 15-20, 20-40, 40-50, 50-100, 100-200, 200-500, and greater than 500. In another embodiment, the method comprises plating differentiated cells, exposing said differentiated cell to an agent that inhibits the activity, expression, or activity and expression of an HDAC, culturing said cells, and identifying a cell that has been reprogrammed.

In another embodiment, the invention relates to a method for reprogramming a cell comprising exposing a cell to an agent that induces the expression, activity, or expression and activity a regulatory protein that inhibits the activity of an HDAC, inducing expression of a pluripotent or multipotent gene; and selecting a cell, wherein differentiation potential has been restored to said cell. The activity or expression of a regulatory protein can be increased by any amount including but not limited to 1-5%, 5-10%, 10-20%, 20-30%, 30-40%, 40-50%, 50-60%, 60-70%, 70-80%, 80-90%, 90-95%, and 95-99%, 99-200%, 200-300%, 300-400%, 400-500% and greater than 500%.

In yet another embodiment, the method further comprises selecting a cell using an antibody directed to a protein or a fragment of a protein coded for by a pluripotent or multipotent gene or a pluripotent surface marker. Any type of antibody can be used including but not limited to a monoclonal, a polyclonal, a fragment of an antibody, a peptide mimetic, an antibody to the active region, and an antibody to the conserved region of a protein

In still another embodiment, the method further comprises selecting a cell using a reporter driven by a pluripotent or multipotent gene or a pluripotent or multipotent surface marker. Any type of reporter can be used including but not limited to a fluorescent protein, green fluorescent protein, cyan fluorescent protein (CFP), a yellow fluorescent protein (YFP), bacterial luciferase, jellyfish aequorin, enhanced green fluorescent protein, chloramphenicol acetyltransferase (CAT), dsRED, β-galactosidase, and alkaline phosphatase.

In still another embodiment, the method further comprises selecting a cell using resistance as a selectable marker including but not limited to resistance to an antibiotic, a fungicide, puromycin, hygromycin, dihydrofolate reductase, thymidine kinase, neomycin resistance (neo), G418 resistance, mycophenolic acid resistance (gpt), zeocin resistance protein and streptomycin.

In still another embodiment, the method further comprises comparing the chromatin structure of a pluripotent or multipotent gene of a cell, prior to exposing said cell to an agent that inhibits the activity, expression or activity and expression of an HDAC, to the chromatin structure of a pluripotent or multipotent gene obtained after treatment with said agent. Any aspect of chromatin structure can be compared including but not limited to euchromatin, heterochromatin, histone acetylation, histone methylation, the presence and absence of histone or histone components, the location of histones, the arrangement of histones, and the presence or absence of regulatory proteins associated with chromatin. The chromatin structure of any region of a gene may be compared including but not limited to an enhancer element, an activator element, a promoter, the TATA box, regions upstream of the start site of transcription, regions downstream of the start site of transcription, exons and introns.

In still another embodiment, the method comprises inhibiting the activity of at least one HDAC, demethylating at least one cytosine in a CpG dinucleotide, and inducing the expression of at least one gene that contributes to a cell being pluripotent or multipotent.

In yet another embodiment, the method comprises contacting a cell with an HDAC inhibitor; inhibiting the activity of an HDAC; and inducing the expression of at least one gene that contributes to a cell being pluripotent or multipotent. In yet another embodiment, the method further comprises producing a reprogrammed cell. The reprogrammed cell can be pluripotent or multipotent.

An HDAC inhibitor of the methods, compositions and kits of the invention may interact with any HDAC. For example, an HDAC inhibitor of the invention may interact with an HDAC from one of the four known classes of HDACs. An HDAC inhibitor of the invention may interact with an HDAC of class I, class II, class III, or class IV. An HDAC inhibitor may interact with one specific class of HDACs, all classes of HDACS, or with multiple classes of HDACs including but not limited class I and class II; class I and class III; class I and class IV; class II and class III; class II and class IV; class III and class IV; class I, II and III; class II, III and IV; and class I, II, III and IV. An HDAC inhibitor may also interact with HDACs that do not fall into one of the known classes.

An HDAC inhibitor may have an irreversible mechanism of action or a reversible mechanism of action. An HDAC inhibitor can have any binding affinity including but not limited to millimolar (mM), micromolar (μM), nanomolar (nM), picomolar (pM), and fentamolar (fM).

Preferably, such inhibition is specific, i.e., the histone deacetylase inhibitor. reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

In another embodiment, the HDAC inhibitor may act by binding to the zinc containing catalytic domain of the HDACs. HDAC inhibitors with this mechanism of action fall into several groupings: (i) hyroxamic acids, such as Trichostatin A; (ii) cyclic tetrapeptides; (iii) benzamides; (iv) electrophilic ketones; and (v) the aliphatic acid group of compounds such as phenylbutyrate and valproic acid.

In yet another embodiment, the HDAC inhibitor can be directed toward the sirtuin Class III HDACs, which are NAD+ dependent and include but are not limited to nicotinamide, derivatives of NAD, dihydrocoumarin, naphthopyranone, and 2-hydroxynaphaldehydes.

In yet another embodiment, the HDAC inhibitor can alter the degree of acetylation of nonhistone effector molecules and thereby increase the transcription of genes. HDAC inhibitors of the methods, compositions, and kits of the invention should not be considered to act solely as enzyme inhibitors of HDACs. A large variety of nonhistone transcription factors and transcriptional co-regulators are known to be modified by acetylation, including but not limited to ACTR, cMyb, p300, CBP, E2F1, EKLF, FEN1, GATA, HNF-4, HSP90, Ku70, NFκB, PCNA, p53, RB, Runx, SF1 Sp3, STAT, TFIIE, TCF, and YY1. The activity of any transcription factor or protein involved in activating transcription, which is acetylated, could be increased with the methods of the invention.

Table I provides a representative list of compounds that can function as an HDAC inhibitor. The reference to “Isotype” in Table I is meant to merely provide insight as to whether the compound has a preference for a particular class of HDAC. Listing a specific isotype or class of HDAC should not be construed to mean that the compound only has affinity for that isotype or class. HDAC inhibitors of the present invention include derivatives and analogues of any HDAC inhibitor herein mentioned.

Butyric acid, or butyrate, was the first HDAC inhibitor to be identified. However, in millimolar concentrations, butyrate may not be specific for HDAC, it also may inhibit phosphorylation and methylation of nucleoproteins as well as DNA methylation. The analogue, phenylbutyrate, acts in a similar manner. More specific are trichostatin A (TSA) and trapoxin (TPX). TPX and TSA have emerged as inhibitors of histone deacetylases. TSA reversibly inhibits, whereas TPX irreversibly binds to and inactivates HDAC enzymes. Unlike butyrate, nonspecific inhibition of other enzyme systems has not yet been reported for TSA or TPX.

Valproic acid also inhibits histone deacetylase activity. VPA is a known drug with multiple biological activities that depend on different molecular mechanisms of action. VPA is an antiepileptic drug. VPA is teratogenic. When used as antiepileptic drug during pregnancy, VPA may induce birth defects (neural tube closure defects and other malformations) in a few percent of born children. In mice, VPA is teratogenic in the majority of mouse embryos when properly dosed. VPA activates a nuclear hormone receptor (PPAR-delta.).

TABLE I
A representative list of compounds that can function as an HDAC
inhibitor.

| | | Affinity | |
|---|---|---|---|
| HDAC Inhibitors | Isotype | Range | Chemical Class |
| Butyrate/Sodium Butyrate | class I/IIa | mM | carboxylate |
| Phenyl Butyrate | | | carboxylate |
| Valproic acid (VPA) | class I/IIa | mM | carboxylate |
| AN-9, Pivaloyloxymethyl | n/a | uM | carboxylate |
| butyrate | | | |
| m-Carboxycinnamic acid | n/a | uM | hydroxamate |
| bishydroxamic acid (CBHA) | | | |
| ABHA | n/a | uM | hydroxamate |
| (azeleic bishydroxamic acid) | | | |
| Oxamflatin | n/a | uM | hydroxamate |
| HDAC-42 | | | hydroxamate |
| SK-7041 | HDAC ½ | nM | hydroxamate |
| DAC60 | | | hydroxamate |
| UHBAs | | | |
| Tubacin | HDAC 6 | | hydroxamate |
| Trapoxin B | | | cyclic peptide/ |
| | | | epoxide |
| A-161906 | n/a | | hydroxamate |
| R306465/JNJ16241199 | HDAC ⅛ | | hydroxamate |
| SBHA (suberic | n/a | uM | hydroxamate |
| bishydroxamate) | | | |
| 3-CI-UCHA | | | |
| ITF2357 | class I/II | nM | hydroxamate |
| PDX-101 | class I/II | uM | hydroxamate |
| Pyroxamide | class I, | uM | hydroxamate |
| | unknown | | |
| | class II | | |
| Scriptaid | n/a | uM | hydroxamate |
| Suberoylanilide hydroxamic | class I/II/IV | uM | hydroxamate |
| acid)/Vorinostat/Zolinza | | | |
| Trichostatin A (TSA) | class I/II | nM | hydroxamate |
| LBH-589 (panobinostat) | class I/II | nM | hydroxamate |
| NVP-LAQ824 | class I/II | nM | hydroxamate |
| Apicidin | HDAC ⅔ | nM | cyclic peptide |
| Depsipeptide/FK- | class I/II | | peptide |
| 228/Romidepsin/FR901228 | | | |
| TPX-HA analogue (CHAP); | | nM | hydroxamate |
| CHAP1, CHAP31, CHAP50 | | | |
| CI-994(N-acetyl dinaline) | HDAC ½ | nM | benzamide |
| MS-275 (same as MS-27- | HDAC 1 | nM | benzamide |
| 275) | | | |
| PCK-101 | | | |
| MGCD0103 | HDAC ½ | nM | benzamide |
| Diallyl disulfide (DADS) | n/a | uM | disulfide |
| Sulforaphane (SFN) | n/a | uM | isothiocyanate |
| Sulforaphene (SFN with a | n/a | uM | isothiocyanate |
| double bond) | | | |
| Erucin | n/a | n/a | isothiocyanate |
| Phenylbutyl isothiocyanate | n/a | uM | isothiocyanate |
| Retinoids | | | |
| SFN-N-acetylcysteine (SFN- | n/a | uM | isothiocyanate |
| NAC) | | | |
| SFN-cysteine (SFN-Cys) | n/a | uM | isothiocyanate |
| Biotin | n/a | n/a | methyl-acceptor |
| Alpha-lipoic acid | n/a | n/a | carboxylate |
| Vit E metabolites | n/a | n/a | |
| Trifluoromethyl ketones | useful | nM | trifluoromethyl |
| | | | ketones |
| Alpha-Ketoamides | | | |
| splitomicin | class III | | |
| LAQ824 | class I/II | nM | hydroxamate |
| SK-7068 | HDAC ½ | nM | hydroxamate |
| Panobinostat | class I/II | nM | hydroxamate |
| Belinostat | class I/II | nM | hydroxamate |

A variety of HDAC inhibitors also are available from Sigma Aldrich (St. Louis, Mo.) including but not limited to APHA Compound; Apicidin; Depudecin; Scriptaid; Sirtinol; and Trichostatin A. Further, additional HDAC inhibitors are available from Vinci-Biochem (Italy) including but not limited to 5-Aza-2′-deoxycytidine; CAY10398; CAY10433; 6-Chloro-2,3,4,9-tetrahydro-1H-carbazole-1-carboxamide; HC Toxin; ITSA1; M344; MC 1293; MS-275; Oxamflatin; PXD101; SAHA; Scriptaid; Sirtinol; Splitomicin. Dexamethasone may also be used in combination with any HDAC inhibitor. For example, a composition comprising dexamethasone and to 5-Aza-2′-deoxycytidine can be used.

Any number, any combination and any concentration of HDAC inhibitors can be used, including but not limited to 1, 2, 3, 4, 5, 6, 7, 8, 9, 10, 11-15, 16-20, and 21-25 HDAC inhibitors. One or more than one family of inhibitory proteins may be inhibited. One or more than one mechanism of inhibition may be used including but not limited to small molecule inhibitors, HDAC inhibitors, shRNA, RNA interference, and small interfering RNA.

In yet another embodiment, the invention relates to a method of reprogramming a cell comprising inhibiting two or more inhibitory proteins that function in a compensatory pathway. In another embodiment, the invention relate to a method of reprogramming a cell comprising inhibiting two or more proteins that function in a redundant pathway. In still another embodiment, the invention relates to a method of reprogramming a cell comprising inhibiting one or HDAC proteins, and inhibiting one or more proteins that functions to compensate for the inhibited HDAC. The inhibition of one inhibitory protein, e.g., an HDAC, can lead to an increase in the expression of one or more other inhibitory proteins. Inhibiting the expression of the redundant, compensatory, or the redundant and compensatory proteins can be accomplished using any suitable method including but not limited to shRNA, RNA interference, HDAC inhibitors, and small molecule inhibitors.

In still another embodiment, the invention relates to methods for reprogramming a cell comprising inhibiting the expression, activity, or the expression and activity of an inhibitory protein, wherein the inhibition of said inhibitory protein does not cause an increase in the expression, activity, or expression and activity of other inhibitory proteins.

In yet another embodiment, the invention relates to a method for reprogramming a cell comprising inhibiting the expression, activity, or the expression and activity of an inhibitory protein, wherein the inhibition of said inhibitory protein does not cause an increase in the expression, activity, or expression and activity of a compensatory protein.

In yet another embodiment, the invention relates to a method for reprogramming a cell comprising inhibiting the expression, activity, or the expression and activity of an inhibitory protein, wherein the inhibition of said inhibitory protein does not cause an increase in the expression, activity, or expression and activity of a redundant protein.

In still another embodiment, the invention relates to a method for reprogramming a cell comprising: exposing a cell to an agent that inhibits that activity, expression or expression and activity of more than one regulatory protein. The regulatory protein can be of the same family or a distinct protein family member. In yet another embodiment, the invention relates to a method for reprogramming a cell comprising: exposing a cell to an agent that inhibits that activity, expression or expression and activity of a first regulatory protein; exposing said cell to a second agent that inhibits the activity, expression or expression and activity of a second regulatory protein, wherein said second regulatory protein has a distinct function from the first regulatory protein. The first and second regulatory proteins can be any protein involved in regulating or altering expression of proteins including but not limited to a histone deacetylase, a histone acetyltransferase, a lysine methyltransferase, a histone methyltransferase, a Trichostatin A, a histone demethylase, a lysine demethylase, a sirtuin, and a sirtuin activator, nuclear receptors, orphan nuclear receptors, Esrrβ and Esrrγ.

A reprogrammed cell produced by the methods of the invention may be pluripotent or multipotent. A reprogrammed cell produced by the methods of the invention can have a variety of different properties including embryonic stem cell like properties. For example, a reprogrammed cell may be capable of proliferating for at least 10, 15, 20, 30, or more passages in an undifferentiated state. In other forms, a reprogrammed cell can proliferate for more than a year without differentiating. Reprogrammed cells can also maintain a normal karyotype while proliferating and/or differentiating. Some reprogrammed cells also can be cells capable of indefinite proliferation in vitro in an undifferentiated state. Some reprogrammed cells also can maintain a normal karyotype through prolonged culture. Some reprogrammed cells can maintain the potential to differentiate to derivatives of all three embryonic germ layers (endoderm, mesoderm, and ectoderm) even after prolonged culture. Some reprogrammed cells can form any cell type in the organism. Some reprogrammed cells can form embryoid bodies under certain conditions, such as growth on media that do not maintain undifferentiated growth. Some reprogrammed cells can form chimeras through fusion with a blastocyst, for example.

Reprogrammed cells can be defined by a variety of markers. For example, some reprogrammed cells express alkaline phosphatase. Some reprogrammed cells express SSEA-1, SSEA-3, SSEA-4, TRA-1-60, and/or TRA-1-81. Some reprogrammed cells express Oct 4, Sox2, and Nanog. It is understood that some reprogrammed cells will express these at the mRNA level, and still others will also express them at the protein level, on for example, the cell surface or within the cell.

A reprogrammed cell can have any combination of any reprogrammed cell property or category or categories and properties discussed herein. For example, a reprogrammed cell can express alkaline phosphatase, not express SSEA-1, proliferate for at least 20 passages, and be capable of differentiating into any cell type. Another reprogrammed cell, for example, can express SSEA-1 on the cell surface, and be capable of forming endoderm, mesoderm, and ectoderm tissue and be cultured for over a year without differentiation.

A reprogrammed cell can be alkaline phosphatase (AP) positive, SSEA-1 positive, and SSEA-4 negative. A reprogrammed cell also can be Nanog positive, Sox2 positive, and Oct-4 positive. A reprogrammed cell also can be Tcl1 positive, and Tbx3 positive. A reprogrammed cell can also be Cripto positive, Stellar positive and Daz1 positive. A reprogrammed cell can express cell surface antigens that bind with antibodies having the binding specificity of monoclonal antibodies TRA-1-60 (ATCC HB-4783) and TRA-1-81 (ATCC HB-4784). Further, as disclosed herein, a reprogrammed cell can be maintained without a feeder layer for at least 10, 11, 12, 13, 14, 15, 16, 17, 18, 19, 20 passages or for over a year.

A reprogrammed cell may have the potential to differentiate into a wide variety of cell types of different lineages including fibroblasts, osteoblasts, chondrocytes, adipocytes, skeletal muscle, endothelium, stroma, smooth muscle, cardiac muscle, neural cells, hemiopoetic cells, pancreatic islet, or virtually any cell of the body. A reprogrammed cell may have the potential to differentiate into all cell lineages. A reprogrammed cell may have the potential to differentiate into any number of lineages including 1, 2, 3, 4, 5, 6-10, 11-20, 21-30, and greater than 30 lineages.

Any gene that contributes to a cell being pluripotent or multipotent may be induced by the methods of the invention including but not limited to glycine N-methyltransferase (Gnmt), Octamer-4 (Oct4), Nanog, SRY (sex determining region Y)-box 2 (also known as Sox2), Myc, REX-1 (also known as Zfp-42), Integrin α-6, Rox-1, LIF-R, TDGF1 (CRIPTO), Fragilis, SALL4 (sal-like 4), GABRB3, LEFTB, NR6A1, PODXL, PTEN, Leukocyte cell derived chemotaxin 1 (LECT1), BUB1, and Krüppel-like factors (KU) such as Klf4 and Klf5. Any number of genes that contribute to a cell being pluripotent or multipotent can be induced by the methods of the invention including but not limited to 1, 2, 3, 4, 5, 6, 7, 8, 9, 10, 11-20, 21-30, 31-40, 41-50, and greater than 50 genes.

Further, Ramalho-Santos et al. (Science 298, 597 (2002)), Ivanova et al. (Science 298, 601 (2002)) and Fortunel et al. (Science 302, 393b (2003)) each compared three types of stem cells and identified a list of commonly expressed “stemness” genes, proposed to be important for conferring the functional characteristics of stem cells. Any of the genes identified in the above-mentioned studies may be induced by the methods of the invention. Table II provides a list of genes thought to be involved in conferring the functional characteristics of stem cells. In addition to the genes listed in Table II, 93 expressed sequence tags (EST) clusters with little or no homology to known genes were also identified by Ramalho-Santos et al. and Ivanova et al, and are included within the methods of the invention.

TABLE II
Genes implicated in conferring stem cell characteristics

| symbol | Gene | Function |
|---|---|---|
| F2r | Thrombin receptor | G-protein coupled receptor, coagulation |
| | | cascade, required for vascular development |
| Ghr | Growth hormone receptor | Growth hormone receptor/binding protein, |
| | | activates Jak2 |
| Itga6 | Integrin alpha 6 | cell adhesion, cell-surface mediated |
| | | signalling, can combine with Integrin b1 |
| Itgb1 | Integrin beta 1 (fibronectin | cell adhesion, cell-surface mediated |
| | Receptor) | signalling, can combine with Integrin a6 |
| Adam 9 | A disintegrin and | cell adhesion, extracellular proteolysis, |
| | metalloproteinase domain 9 | possible fusogenic function |
| | (meltrin gamma) | |
| Bys | Bystin-like (Bystin) | cell adhesion, may be important for embryo |
| | | implantation (placenta) |
| Ryk | Receptor-like tyrosine kinase | unconventional receptor tyrosine kinase |
| Pkd2 | Polycystic kidney disease 2 | calcium channel |
| Kcnab3 | Potassium voltage gated | Regulatory subunit of potassium channel |
| | channel, shaker related | |
| | subfamily, beta member 3 | |
| Gnb1 | Guanine nucleotide binding | G-protein coupled receptor signaling |
| | protein beta 1 | |
| Gab1 | Growth factor receptor | integration of multiple signaling pathways |
| | bound protein 2 (Grb2)- | |
| | associated protein 1 | |
| Kras2 | Kirsten rat sarcoma | binds GTP and transmits signals from growth |
| | oncogene 2 | factor receptors |
| | ESTs highly similar to Ras | suppressor of RAS function |
| | p21 protein activator (Gap) | |
| Cttn | Cortactin | regulates actin cytoskeleton, overexpressed in |
| | | tumors |
| Cops4 | COP9 (constitutive | Cop9 signalosome, integration of multiple |
| | photomorphogenic), subunit | signaling pathways, regulation of protein |
| | 4 | degradation |
| Cops7a | COP9 (constitutive | Cop9 signalosome, integration of multiple |
| | photomorphogenic), subunit | signaling pathways, regulation of protein |
| | 7a | degradation |
| Madh1 | Mad homolog 1 (Smad1) | TGFb pathway signal transducer |
| Madh2 | Mad homolog 2 (Smad2) | TGFb pathway signal transducer |
| Tbrg1 | TGFb regulated 1 | induced by TGFb |
| Stam | signal transducing adaptor | Associates with Jak tyrosine kinase |
| | molecule (SH3 domain and | |
| | ITAM motif) 1 | |
| Statip1 | STAT interacting protein 1 | scaffold for Jak/Stat3 binding |
| Cish2 | Cytokine inducible SH2- | STAT induced STAT inhibitor-2, interacts |
| | containing protein 2 (Ssi2) | with IgflR |
| | ESTs moderately similar to | possible tyrosine kinase |
| | Jak3 | |
| | ESTs highly similar to | regulatory subunit of protein phosphatase 2, |
| | PPP2R1B | putative tumor suppressor |
| Rock2 | Rho-associated coiled-coil | serine/theonine kinase, target of Rho |
| | forming kinase 2 | |
| Yes | Yamaguchi sarcoma viral | intracellular tyrosine kinase, proto-oncogene, |
| | oncogene homolog | Src family |
| Yap | Yes-associated protein 1 | bind Yes, transcriptional co-activator |
| Ptpn2 | Protein tyrosine non-receptor | dephosphorylates proteins |
| | phosphatase 2 | |
| Ppplr2 | Protein phosphatase 1, | Inhibitory subunit of protein phosphatase 1 |
| | regulatory (inhibitor) 2 | |
| Ywhab | Tyrosine/tryptophan | Binds phosphoserine-proteins, PKC pathway |
| | monooxgenase activation | |
| | protein beta (14-3-3beta) | |
| Ywhah | Tyrosine/tryptophan | Binds phosphoserine-proteins, PKC pathway |
| | monooxgenase activation | |
| | protein eta (14-3-3eta) | |
| Axo | Axotrophin | contains a PHD domain, an adenylaye cyclase |
| | | domain and a consensus region for G-protein |
| | | interaction, required for neuronal |
| | | maintenance |
| Trip6 | Thyroid hormone receptor | interacts with THR in the presence of TH, |
| | interactor 6 | putative co-activator for Rel transcription |
| | | factor |
| Gfer | Growth factor, erv1 (S. | sulphydryl oxidase, promotes liver |
| | cerevisiae)-like (augmenter | regeneration, stimulates EGFR and MAPK |
| | of liver regeneration) | pathways |
| Upp | Uridine phosphorylase | Interconverts uridine and uracil, highly |
| | | expressed in transformed cells, may produce |
| | | 2-deoxy-D-ribose, a potent angiogenic factor |
| Mdfi | MyoD family inhibitor | inhibitor of bHLH and beta-catenin/TCF |
| | | transcription factors |
| Tead2 | TEA domain 2 | transcriptional factor |
| Yap | Yes-associated 65 kD | Binds Yes, transcriptional co-activator |
| Fhl1 | Four and a half LIM | may interact with RBP-J/Su(H) |
| Zfx | Zinc Finger X-linked | zinc finger, putative transcription factor |
| Zfp54 | Zinc finger 54 | zinc finger, putative transcription factor |
| | Zinc finger protein | zinc finger, putative transcription factor |
| D17Ertd197e | D17Ertd197e | zinc finger, putative transcription factor |
| | ESTs, high similarity to Zfp | zinc finger, putative transcription factor |
| | ESTs, high similarity to Zfp | zinc finger, putative transcription factor |
| | ESTs, high similarity to Zfp | zinc finger, putative transcription factor |
| Rnf4 | RING finger 4 | steroid-mediated transcription |
| Chd1 | Chromodomain helicase | modification of chromatin structure, |
| | DNA binding protein 1 | SNF2/SW12 family |
| Etl1 | enhancer trap locus 1 | modification of chromatin structure, |
| | | SNF2/SW12 family |
| Rmp | Rpb5-mediating protein | Binds RNA, PolII inhibits transcription |
| Ercc5 | Excision repair 5 | Endonuclease, repair of UV-induced damage |
| Xrcc5 | X-ray repair 5 (Ku80) | helicase, involved in V(D)J recombination |
| Msh2 | MutS homolog 2 | mismatch repair, mutated in colon cancer |
| Rad23b | Rad23b homolog | excision repair |
| Ccnd1 | Cyclin D1 | G1/S transition, regulates CDk2 and 4, |
| | | overexpressed in breast cancer, implicated in |
| | | other cancers |
| Cdkn1a | Cdk inhibitor 1a P21 | inhibits G1/S transition, Cdk2 inhibitor, |
| | | required for HSC maintenance |
| Cdkap1 | Cdk2 associated protein | binds DNA primase, possible regulator of |
| | | DNA replication (S phase) |
| Cpr2 | Cell cycle progression 2 | overcomes G1 arrest in S. cerevisiae |
| Gas2 | Growth arrest specific 2 | highly expressed in growth arrested cells, part |
| | | of actin cytoskeleton |
| CenpC | Centromere protein C | present in active centromeres |
| Wig1 | Wild-type p53 induced 1 | p53 target, inhibits tumor cell growth |
| Tmk | Thymidylate kinase | dTTP synthesis pathway, essential for S phase |
| | | progression |
| Umps | Uridine monophosphate | Pyrimidine biosynthesis |
| | synthetase | |
| Sfrs3 | Splicing factor RS rich 3 | implicated in tissue-specific differential |
| | | splicing, cell cycle regulated |
| | ESTs highly similar to | Cell cycle-regulated nuclear export protein |
| | exportin 1 | |
| | ESTs highly similar to CAD | trifunctional protein of pyrimidine |
| | | biosynthesis, activated (phosphorylated) by |
| | | MAPK |
| | ESTs similar to Mapkkkk3 | Map kinase cascade |
| Gas2 | Growth arrest specific 2 | highly expressed in growth arrested cells, part |
| | | of actin cytoskeleton, target of caspase-3, |
| | | stabilizes p53 |
| Wig1 | Wild-type p53 induced 1 | p53 target, inhibits tumor cell growth |
| Pdcd2 | Programmed cell death 2 | Unknown |
| Sfrs3 | Splicing factor RS rich 3 | implicated in tissue-specific differential |
| | | splicing, cell cycle regulated |
| | ESTs highly similar to Sfrs6 | putative splicing factor |
| | ESTs highly similar to pre- | putative splicing factor |
| | mRNA splicing factor Prp6 | |
| Snrp1c | Small nuclear | U1 snRNPs, component of the spliceosome |
| | ribonucleoprotein | |
| | polypeptide C | |
| Phax | Phosphorylated adaptor for | mediates U snRNA nuclear export |
| | RNA export | |
| NOL5 | Nucleolar protein 5 (SIK | pre-rRNA processing |
| | similar) | |
| | ESTs highly similar to | pre-rRNA processing |
| | Nop56 | |
| Rnac | RNA cyclase | Unknown |
| | ESTs highly similar to Ddx1 | DEAD-box protein, putative RNA helicase |
| Eif4ebp1 | Eukaryotic translation | translational repressor, regulated |
| | initiation factor 4E binding | (phosphorylated) by several signaling |
| | protein 1 | pathways |
| Eif4g2 | Eukaryotic translation | translational repressor, required for |
| | initiation factor 4, gamma 2 | gastrulation and ESC differentiation |
| | ESTs highly similar to | Translation initiation factor |
| | Eif3s1 | |
| Mrps31 | Mitochondrial ribosomal | component of the ribosome, mitochondria |
| | protein S31 | |
| Mrpl17 | Mitochondrial ribosomal | component of the ribosome, mitochondria |
| | protein L17 | |
| Mrpl34 | Mitochondrial ribosomal | component of the ribosome, mitochondria |
| | protein L34 | |
| Hspa11 | Heat shock 70 kD protein- | Chaperone, testis-specific |
| | like 1 (Hsc70t) | |
| Hspa4 | Heat shock 70 kDa protein 4 | Chaperone |
| | (Hsp110) | |
| Dnajb6 | DnaJ (Hsp40) homolog, | co-chaperone |
| | subfamily B, member 6 | |
| | (Mammalian relative of | |
| | Dnaj) | |
| Hrsp12 | Heat responsive | possible chaperone |
| Tcp1-rs1 | T-complex protein 1 related | possible chaperone |
| | sequence 1 | |
| Ppic | Peptidylprolyl isomerase C | Isomerization of peptidyl-prolyl bonds |
| | (cyclophilin C) | |
| Fkbp9 | FK506-binding protein 9 | possible peptidyl-prolyl isomerase |
| | (63 kD) | |
| | ESTs moderately similar to | possible peptidyl-prolyl isomerase |
| | Fkbp13 | |
| Ube2d2 | Ubiquitin-conjugating | E2, Ubiquitination of proteins |
| | enzyme E2D2 | |
| Arih1 | Ariadne homolog | likely E3, Ubiquitin ligase |
| Fbxo8 | F-box only 8 | putative SCF Ubiquitin ligase subunit |
| | ESTs moderately similar to | possible E2, Ubiquitination of proteins |
| | Ubc13 (bendless) | |
| Usp9x | Ubiquitin protease 9, X | removes ubiquitin from proteins |
| | chromosome | |
| Uchrp | Ubiquitin c-terminal | likely removes ubiquitin from proteins |
| | hydrolase related | |
| | polypeptide | |
| Axo | Axotrophin | contains RING-CH domain similar to E3s, |
| | | Ubiquitin ligases |
| Tpp2 | Tripeptidyl peptidase II | serine expopeptidase, associated with the |
| | | proteasome |
| Cops4 | COP9 (constitutive | Cop9 signalosome, integration of multiple |
| | photomorphogenic) subunit | signaling pathways, regulation of protein |
| | 4 | degredation |
| Cops 7a | COP9 (constitutive | Cop9 signalosome, integration of multiple |
| | photomorphogenic), subunit | signaling pathways, regulation of protein |
| | 7a | degradation |
| | ESTs highly similar to | Regulatory subunit of the proteasome |
| | proteasome 26S subunit, | |
| | non-ATPase, 12 (p55) | |
| Nyren18 | NY-REN-18 antigen | interferon-9 induced, downregulator of |
| | (NUB1) | Nedd8, a ubiquitin-like protein |
| Rab18 | Rab18, member RAS | small GTPase, may regulate vesicle transport |
| | oncogene family | |
| Rabggtb | RAB geranlygeranyl | regulates membrane association of Rab |
| | transferase, b subunit | proteins |
| Stxbp3 | Syntaxin binding protein 3 | vesicle/membrane fusion |
| Sec23a | Sec23a (S. cerevisiae) | ER to Golgi transport |
| | ESTs moderately similar to | ER to Golgi transport |
| | Coatomer delta | |
| Abcb1 | Multi-drug resistance 1 | exclusion of toxic chemicals |
| | (Mdr1) | |
| Gsta4 | Glutathione S-transferase 4 | response to oxidative stress |
| Gslm | Glutamate-cycteine ligase | glutathione biosynthesis |
| | modifier subunit | |
| Txnrd1 | Thioredoxin reductase | delivers reducing equivalents to Thioredoxin |
| Txn1 | Thioredoxin-like 32 kD | redox balance, reduces dissulphide bridges in |
| | | proteins |
| Laptm4a | Lysosomal-associated | import of small molecules into lysosome |
| | protein transmembrane 4A | |
| | (MTP) | |
| Rcn | Reticulocalbin | ER protein, Ca+2 binding, overexpressed in |
| | | tumor cell lines |
| Supl15h | Suppressor of Lec15 | ER synthesis of dolichol phosphate-mannose, |
| | homolog | precursor to GPI anchors and N-glycosylation |
| Pla2g6 | Phospholipase A2, group VI | Hydrolysis of phospholipids |
| Acadm | Acetyl-Coenzyme A | fatty acid beta-oxidation |
| | dehydrogenase, medium | |
| | chain | |
| Suclg2 | Succinate-Coenzyme A | regulatory subunit, Krebs cycle |
| | ligase, GDP-forming, beta | |
| | subunit | |
| Pex7 | Peroxisome biogenesis | Peroxisomal protein import receptor |
| | factor 7 | |
| Gcat | Glycine C-acetyltransferase | conversion of threonine to glycine |
| | (KBL) | |
| Tjp1 | Tight junction protein 1 | component of tight junctions, interacts with |
| | | cadherins in cells lacking tight junctions |

Embodiments of the invention also relate to methods for reprogramming a cell comprising modifying chromatin structure of a gene, and inducing the expression of said gene. In another embodiment, the method comprises modifying the chromatin structure of a pluripotent or multipotent gene. In still yet another embodiment, the method further comprises modifying the chromatin structure by modifying a histone. Modifying a histone includes but is not limited to acetylation; methylation; demethylation; phosphorylation; ubiquitination; sumoylation; ADP-ribosylation; deimination and proline isomerization.

Embodiments of the invention also include methods for treating a variety of diseases using a reprogrammed cell produced according to the methods disclosed herein. The skilled artisan would appreciate, based upon the disclosure provided herein, the value and potential of regenerative medicine in treating a wide plethora of diseases including, but not limited to, heart disease, diabetes, skin diseases and skin grafts, spinal cord injuries, Parkinson's disease, multiple sclerosis, Alzheimer's disease, and the like. The invention encompasses methods for administering reprogrammed cells to an animal, including humans, in order to treat diseases where the introduction of new, undamaged cells will provide some form of therapeutic relief.

The skilled artisan will readily understand that reprogrammed cells can be administered to an animal as a re-differentiated cell, for example, a neuron, and will be useful in replacing diseased or damaged neurons in the animal. Additionally, a reprogrammed cell can be administered to the animal and upon receiving signals and cues from the surrounding milieu, can re-differentiate into a desired cell type dictated by the neighboring cellular milieu. Alternatively, the cell can be re-differentiated in vitro and the differentiated cell can be administered to a mammal in need there of.

The reprogrammed cells can be prepared for grafting to ensure long term survival in the in vivo environment. For example, cells can be propagated in a suitable culture medium, such as progenitor medium, for growth and maintenance of the cells and allowed to grow to confluence. The cells are loosened from the culture substrate using, for example, a buffered solution such as phosphate buffered saline (PBS) containing 0.05% trypsin supplemented with 1 mg/ml of glucose; 0.1 mg/ml of MgCl.sub.2, 0.1 mg/ml CaCl.sub.2 (complete PBS) plus 5% serum to inactivate trypsin. The cells can be washed with PBS using centrifugation and are then resuspended in the complete PBS without trypsin and at a selected density for injection.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The invention also encompasses grafting reprogrammed cells in combination with other therapeutic procedures to treat disease or trauma in the body, including the CNS, PNS, skin, liver, kidney, heart, pancreas, and the like. Thus, reprogrammed cells of the invention may be co-grafted with other cells, both genetically modified and non-genetically modified cells which exert beneficial effects on the patient, such as chromaffin cells from the adrenal gland, fetal brain tissue cells and placental cells. Therefore the methods disclosed herein can be combined with other therapeutic procedures as would be understood by one skilled in the art once armed with the teachings provided herein.

The reprogrammed cells of the invention can be transplanted “naked” into patients using techniques known in the art such as those described in U.S. Pat. Nos. 5,082,670 and 5,618,531, each incorporated herein by reference, or into any other suitable site in the body.

The reprogrammed cells can be transplanted as a mixture/solution comprising of single cells or a solution comprising a suspension of a cell aggregate. Such aggregate can be approximately 10-500 micrometers in diameter, and, more preferably, about 40-50 micrometers in diameter. A reprogrammed cell aggregate can comprise about 5-100, more preferably, about 5-20, cells per sphere. The density of transplanted cells can range from about 10,000 to 1,000,000 cells per microliter, more preferably, from about 25,000 to 500,000 cells per microliter.

Transplantation of the reprogrammed cell of the present invention can be accomplished using techniques well known in the art as well those developed in the future. The invention comprises a method for transplanting, grafting, infusing, or otherwise introducing reprogrammed cells into an animal, preferably, a human.

The reprogrammed cells also may be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

Reprogrammed cells of the present invention can also be used to express a foreign protein or molecule for a therapeutic purpose or for a method of tracking their integration and differentiation in a patient's tissue. Thus, the invention encompasses expression vectors and methods for the introduction of exogenous DNA into reprogrammed cells with concomitant expression of the exogenous DNA in the reprogrammed cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Embodiments of the invention also relate to a composition comprising a cell that has been produced by the methods of the invention. In another embodiment, the invention relates to a composition comprising cell that has been reprogrammed by inhibiting the activity of at least one HDAC. In yet another embodiment, the invention relates to a composition comprising a cell that has been reprogrammed by inducing the expression of at least one gene that contributes to a cell being pluripotent or multipotent.

Embodiments of the invention also relate to a reprogrammed cell that has been produced by contacting a cell with at least one HDAC inhibitor.

Embodiments of the invention also relate to kits for preparing the methods and compositions of the invention. The kit can be used for, among other things, producing a reprogramming a cell and generating ES-like and stem cell-like cells, inducing the expression of at least one gene that contributes to a cell being pluripotent or multipotent, and inhibiting the activity of at least one HDAC. The kit may comprise at least one HDAC inhibitor. The kit may comprise multiple HDAC inhibitors. The HDAC inhibitors can be provided in a single container or in multiple containers.

The kit may also comprise reagents necessary to determine if the cell has been reprogrammed including but not limited to reagents to test for the induction of a gene that contributes to a cell being pluripotent or multipotent, reagents to test for inhibition of an HDAC, and regents to test for remodeling the chromatin structure.

The kit may also comprise regents that can be used to differentiate the reprogrammed cell into a particular lineage or multiple lineages including but not limited to a neuron, an osteoblast, a muscle cell, an epithelial cell, and hepatic cell.

The kit may also contain an instructional material, which describes the use of the components provide in the kit. As used herein, an “instructional material” includes a publication, a recording, a diagram, or any other medium of expression that can be used to communicate the usefulness of the methods of the invention in the kit for, among other things, effecting the reprogramming of a differentiated cell. Optionally, or alternately, the instructional material may describe one or more methods of re- and/or trans-differentiating the cells of the invention. The instructional material of the kit of the invention may, for example, be affixed to a container that contains the HDAC inhibitor. Alternatively, the instructional material may be shipped separately from the container with the intention that the instructional material and the HDAC inhibitor, or component thereof, be used cooperatively by the recipient.

The invention is now described with reference to the following Examples. These Examples are provided for the purpose of illustration only and the invention should in no way be construed as being limited to these Examples, but rather should be construed to encompass any and all variations that become evident as a result of the teaching provided herein. All references including but not limited to U.S. patents, allowed U.S. patent applications, or published U.S. patent applications are incorporated within this specification by reference in their entirety.

### EXAMPLES

The following examples are illustrative only and are not intended to limit the scope of the invention as defined by the claims.

### Example 1

Histone deacetylase inhibitors have been shown to acetylate histone proteins and demethylate DNA, thereby modifying chromatin structure in at least two ways. The expression level of genes that contribute to a cell being pluripotent was tested in the presence and absence of a histone deacetylase inhibitor. In the present example, valproic acid (VPA) was used but any histone deaceytlase inhibitor can be used.

Methods

Cell culture. Primary human lung cells were purchased from Cell Applications (San Diego, Calif.), and were maintained at 37° C. in 95% humidity and 5% CO₂ in Dulbecco's modified eagle medium (DMEM, Hyclone) containing 10% fetal bovine serum (FBS, Hyclone) and 0.5% penicillin and streptomycin. Cells were grown in the presence of 1 mM VPA, 5 mM VPA or in the absence of VPA for three days.

Quantitative RT-PCR. Expression of Oct-4 and Nanog were determined by real-time RT-PCR for each culture condition (0 mM VPA, 1 mM VPA, and 5 mM VPA). Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of Oct-4 and Nanog was normalized to GAPD.

Embryonic Taqman Low Density Array Analysis. Expression levels of several genes that contribute to a cell being pluripotentail (“stemness genes”) were determined using the Human Embryonic Taqman Low Density Array Analysis (TLDA). Several stemness genes were analyzed: GABRB3, LEFTB, NR6A1, PODXL, and PTEN. In addition, the expression level of the DNA methyl transferase DNMT3B was determined. The Applied Biosystems Human Embryonic TLDA, which contains 90 embryonic stem cell and developmental genes and 6 endogenous control genes, was used for quantitative real time RT-PCR to quantify relative expression levels (Applied Biosytems, Foster City, Calif.). Briefly, following reverse-transcription of RNA using the ABI High Capacity cDNA Reverse Transcription Kit (ABI; Foster City, Calif.), 150 ng sample cDNA in 50 μl nuclease-free water+50 μl ABI Universal Taqman 2×PCR Master Mix was pipetted into each port of the TLDA microfluidic card, and analyzed on the ABI 7900HT Fast Real Time PCR System. The ^{ΔΔ}CT method was used to calculate relative quantities (fold change) in gene expression levels in treated cells relative to untreated control cells. The treated cells may also be compared to federally-approved human embryonic stem cells.

Bisulfite Sequencing. Bisulfite sequencing is the use of bisulfite treatment of DNA to determine the pattern of methylation. Bisulfite sequencing is based on the fact that treatment of DNA with bisulfite converts cytosine residues to uracil, but leaves 5-methylcytosine residues unaffected. Bisulfite treatment thus introduces specific changes in the DNA sequence that depend on the methylation status of individual cytosine residues, yielding very high-resolution information about the methylation status of a segment of DNA.

Methylation of pluripotent gene promoters was analyzed by bisulfite sequencing. Briefly, DNA was purified by phenolchloroform-isoamylalcohol extraction. Bisulfite conversion was performed using the EZ DNA Methylation kit following the manufacturer's protocol (Zymo Research; Orange, Calif.). The conversion rate of all cytosines in non-CpG dinucleotides to uracils was 100%. Converted DNA was amplified by PCR using primers for human Oct3/4, Nanog, and SOX2. PCR products were cloned into E. coli by TOPO TA cloning kit (Invitrogen; Carlsbad, Calif.). Ten clones of each sample were verified by sequencing with SP6 and T7 primers. The global methylation percentage for each promoter of interest and the number of methylated cytosines for a given CpG was compared among cell populations.

Results

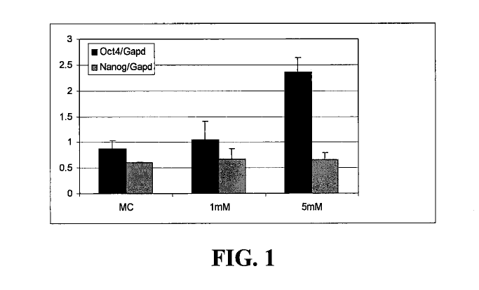

As shown in FIG. 1, the expression of Oct4 was up-regulated (˜2.7-fold; p<0.01) in primary human lung cells treated with 5 mM VPA compared to control cells (MC). These results demonstrate that an HDAC inhibitor can lead to the induction or increased expression of a gene that contributes to a cell being pluripotent.

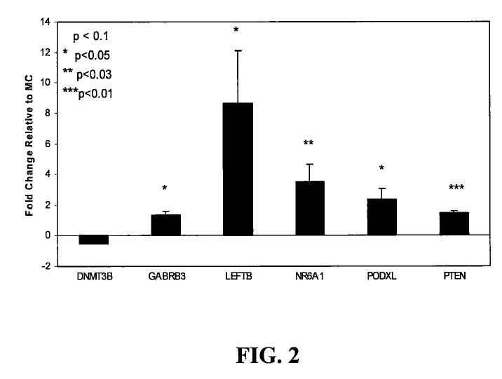

The expression level of several “stemness” genes also was analyzed using cells grown in 5 mM VPA for three days. As shown in FIG. 2, Embryonic Taqman Low Density Array analysis revealed up-regulation of the following stemness genes: GABRB3 (p<0.05); LEFTB (p<0.05); NR6A1 ((p<0.03); PODXL (p<0.05); PTEN (p<0.01) (n=three replicates per group). In addition, the DNA methyltransferase, DNMT3B, was down-regulated. Several other stemness-related genes that were not detected in control cells were induced in the VPA-treated cells, including FOXD3, NR5A2, TERT, LIFR, SFRP2, TFCP2L1, LIN28, SOX2 and XIST.

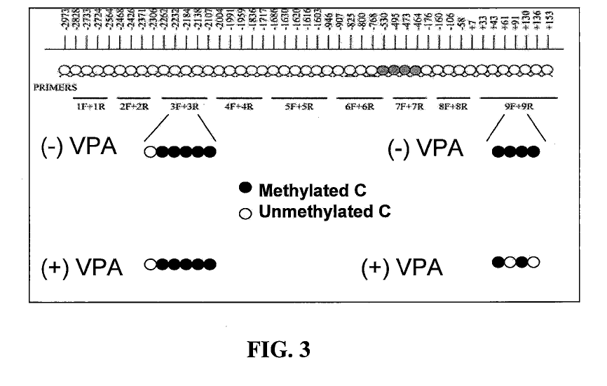

The first exon of the Oct-4 gene was analyzed by bisulfite sequencing. Bisulfite sequencing revealed methylated cytosines in untreated (−) and treated (+) cells upstream from Oct4 (3F-3R) (see FIG. 3). In addition, two cytosines in CpG dinucleotides in the promoter/first exon region of Oct4 in treated cells were demethylated (see FIG. 3). These patterns were consistent among several clones (data not shown).

These results demonstrate that an HDAC inhibitor can induce the expression of genes that contribute to a cell being pluripotent or multipotent, can reduce the expression of a DNA methyl transferase, and de-methylate cytosines in DNA. Additionally, the HDAC inhibitor can lead to demethylation of cytosines in promoter regions of genes that contribute to a cell being pluripotent or multipotent.

### Example 2

The effect of HDAC7 shRNA lentiviral infection on the level of mRNA expression on Oct-4., Nanog, and Sox 2 was tested. In addition, in a separate set of experiments, the effect of HDAC11 shRNA lentiviral infection on the level of mRNA expression on Oct-4., Nanog, and Sox 2 also was tested. Three types of human dermal fibroblasts were used: adult human dermal fibroblasts (HDFa), neonatal human dermal fibroblasts (HDFn), and fetal human dermal fibroblasts (HDFf).

Methods:

Human dermal fibroblasts (HDFa, HDFn, and HDFf) were infected with shRNA lentivirus to interfere with HDAC7. In a separate set of experiments, human dermal fibroblasts (HDFa, HDFn, and HDFf) were infected with shRNA lentivirus to interfere with HDAC11. RNA was isolated from HDFs (including puromycin selection) and applied to RT-PCR to analyze expression of target genes, e.g., Oct-4, Nanog, Sox2, various HDACs and various SIRT genes. The shRNA construct included puromycin (antibiotic) resistance as a way to select cells that have been successfully transfected with the shRNA. After transfection, puromycin was added to the culture and cells that were not resistant (therefore not transfected) died, thereby leaving only transfected cells remaining in the culture.

Cell culture. Human dermal fibroblasts were purchased from Cell Applications (San Diego, Calif.), and were maintained at 37° C. in 95% humidity and 5% CO₂ in Fibroblast growth medium (Cell Applications, San Diego, Calif.).

Lentiviral Infection. Human dermal fibroblasts were infected with a shRNA construct. The shRNA construct was obtained from Dharmacon. The shRNA construct directed toward directed toward HDAC7a had the following sequence:

SEQ ID NO. 1: GCTTTCAGGATAGTCGTGA

An shRNA construct with the following sequence was directed against HDAC11:

SEQ ID NO. 2: AGCGAGACTTCATGGACGA

In addition, an shRNA construct with the following sequence was directed against HDAC11:

SEQ ID. NO. 3: TGGTGGTATACAATGCAGG

The human dermal fibroblasts were infected with the shRNA following the manufacturer's instructions. HDF were cultured with an without puromycin selection and hES culture conditions (mTeSR Medium, Stem Cell Technology, Vancouver, BC, Canada) on matrigel (BD Biosciences, San Jose Calif.). In these sets of experiments, cells were infected with a shRNA construct directed toward either HDAC7, or HDAC11.

Quantitative RT-PCR. Expression of Oct-3/4 and Nanog was determined by real-time RT-PCR. Briefly, total RNA was prepared from cultures using Trizol Reagent (Life Technologies, Gaithersburg, Md.) and RNeasy Mini kit (Qiagen; Valencia, Calif.) with DNase digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of Oct-3/4 and Nanog was normalized to glyceraldehyde 3-phosphate-dehydrogenase (GAPD).

Results:

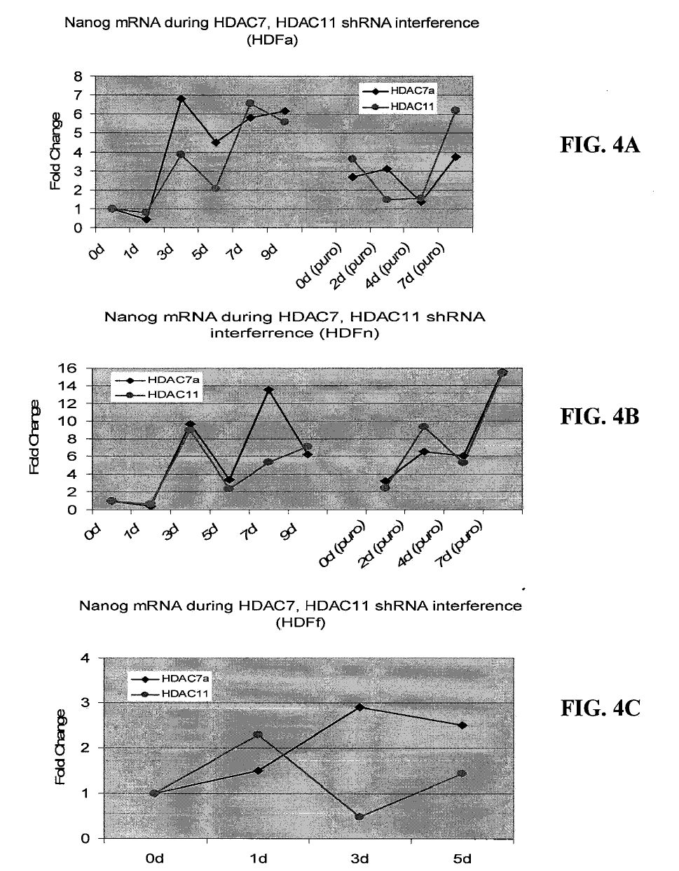

The effects of HDAC7 and HDAC11 shRNA lentiviral infection on the mRNA level of the gene Nanog are shown in FIG. 4A (HDFa), FIG. 4B (HDFn) and FIG. 4C (HDFf). For all three cell types, both HDAC7 and HDAC11 knockdown increased the level of mRNA for the gene Nanog, both in the presence and absence of puromycin (shown for adult and neonatal human dermal fibroblasts). For the cell types HDFa and HDFn, expression of Nanog increased at least six-fold over time. The increase in the level of Nanog mRNA was seen with and without puromycin selection. As reported in FIG. 4A, interference with HDAC7 resulted in a rapid increase in mRNA expression of Nanog as compared to interference with HDAC11. However, with additional time, the increase in the level of mRNA of the gene Nanog appeared to be equal, regardless of whether HDAC7 or HDAC11 was interfered. An increase in the level of mRNA for the gene Nanog was seen in HDFf, but not as robustly as observed for HDFa and HDFn.

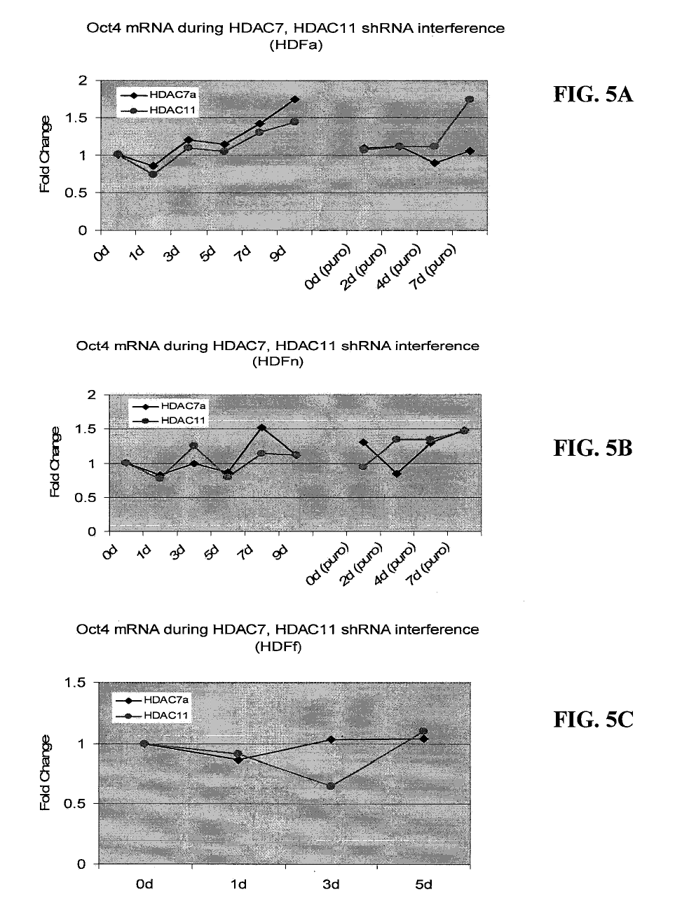

The effects of HDAC7 and HDAC 11 shRNA lentiviral infection on the mRNA level of the gene. Oct-4 are shown in FIG. 5A (HDFa), FIG. 5B (HDFn) and FIG. 5C (HDFf). Both HDAC7 and HDAC11 knockdown increased the level of mRNA for the gene Nanog in the cell types HDFa and HDFn. The increase in expression of Oct-4 was observed both in the presence and absence of puromycin (FIG. 5A and FIG. 5B). A more modest increase in the level of mRNA for the gene Oct-4 was observed as compared to the gene Nanog.

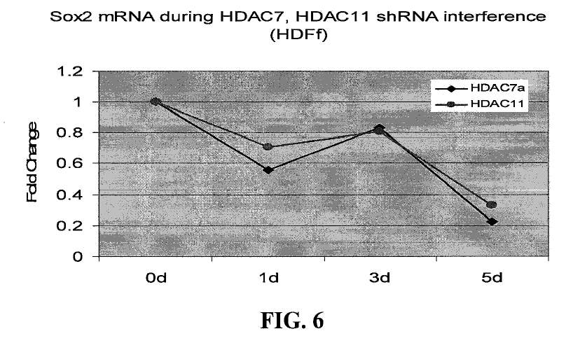

FIG. 6 reports the effect of HDAC7 and HDAC11 shRNA lentiviral infection on the mRNA level of Sox-2 in fetal human dermal fibroblasts. No induction in the level of mRNA for the Sox-2 gene was observed.

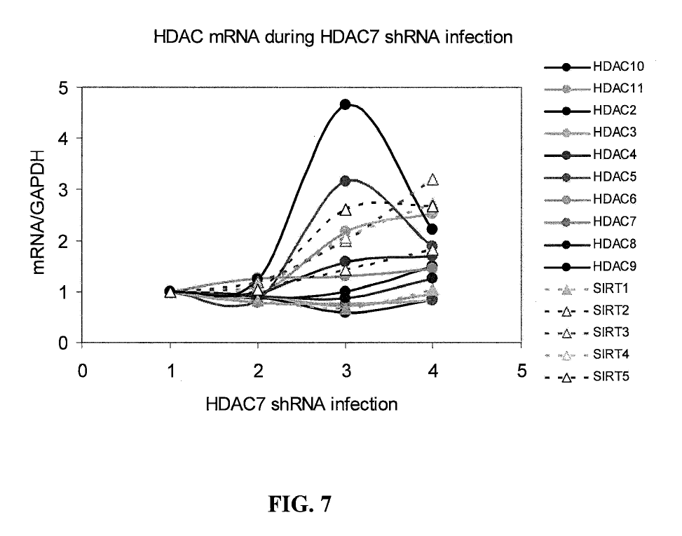

FIG. 7 reports the effects of HDAC7 shRNA lentiviral infection on the level of mRNA expression of various HDAC genes and SIRT genes. As shown in FIG. 7, the expression of HDAC 9, HDAC5 and HDAC11 mRNA was inducted three days after HDAC7 shRNA infection. The level of HDAC7 mRNA was reduced about 50% of basal level around three days after lentiviral infection.

The inhibition of one HDAC, in this case HDAC7, led to an increase in the expression of several other HDAC genes. HDACs are closely related, and have likely evolved to have redundant or at least similar functions. If one family member is inhibited, the expression of other family members may be increased to compensate for the inhibited member. HDACs play a crucial function and therefore, redundant and/or compensatory pathways may have evolved. One mechanism to reprogram a cell may be to simultaneously or sequentially target multiple family members to account for the redundant and/or compensatory pathways. Another mechanism to reprogram a cell may be to simultaneously or sequentially target inhibitory proteins in the same family or to target inhibitory proteins in different families of regulatory proteins.

### Example 3

The effect of HDAC7 and HDAC11 shRNA lentiviral infection on the level of mRNA expression on Oct-4, Nanog, and Sox 2 was tested. In the same experiment, HDAC7 and HDAC11 were interfered with and the effect on the expression of various genes determined. Three types of human dermal fibroblasts were used: adult human dermal fibroblasts (HDFa), neonatal human dermal fibroblasts (HDFn), and fetal human dermal fibroblasts (HDFf).

Methods:

Human dermal fibroblasts (HDFa, HDFn, and HDFf) were infected with shRNA lentivirus to interfere with HDAC7 and HDAC11. RNA was isolated from HDFs (including puromycin selection) and applied to RT-PCR to analyze expression of target genes, e.g., Oct-4, Nanog, Sox2, various HDACs and various SIRT genes. The shRNA construct included puromycin (antibiotic) resistance as a way to select cells that have been successfully transfected with the shRNA. After transfection, puromycin was added to the culture and cells that were not resistant (therefore not transfected) died, thereby leaving only transfected cells remaining in the culture.

Cell culture. Human dermal fibroblasts were purchased from Cell Applications (San Diego, Calif.), and were maintained at 37° C. in 95% humidity and 5% CO₂ in Fibroblast growth medium (Cell Applications, San Diego, Calif.).

Lentiviral Infection. Human dermal fibroblasts were infected with a shRNA construct. The shRNA construct was obtained from Dharmacon. The shRNA construct directed toward directed toward HDAC7a had the following sequence:

SEQ ID NO. 1: GCTTTCAGGATAGTCGTGA

An shRNA construct with the following sequence was directed against HDAC11:

SEQ ID NO. 2: AGCGAGACTTCATGGACGA

In addition, an shRNA construct with the following sequence was directed against HDAC11:

SEQ ID. NO. 3: TGGTGGTATACAATGCAGG

The human dermal fibroblasts were infected with the shRNA following the manufacturer's instructions. HDF were cultured with an without puromycin selection and hES culture conditions (mTeSR Medium, Stem Cell Technology, Vancouver, BC, Canada) on matrigel (BD Biosciences, San Jose Calif.).

Quantitative RT-PCR. Expression of Oct-3/4 and Nanog was determined by real-time RT-PCR. Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of Oct-3/4, Nanog and Sox-2 was normalized to glyceraldehyde 3-phosphate-dehydrogenase (CAPD).

Results:

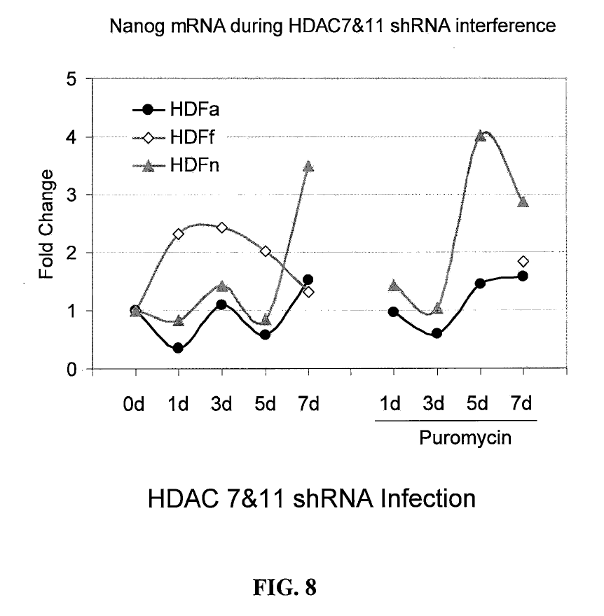

As reported in FIG. 8, Nanog expression increased by double knockdown of HDAC7 and HDAC11 for both cell types HDFf and HDFn, both in the presence and absence of puromycin. Nanog expression increased rapidly in the cell type HDFf and a consistent response was observed through day five. A modest effect was observed in cell type HDFa.

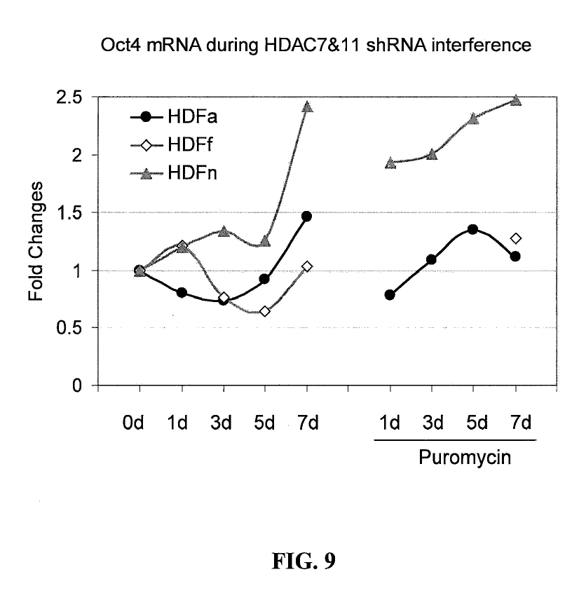

FIG. 9 reports the effect on the mRNA expression of Oct-4 during dual or simultaneous HDAC7 and HDAC11 shRNA interference. The increase in Oct-4 expression was observed both in the presence and absence of puromycin. A robust effect was observed for the cell type HDFn, and the mRNA expression was increased for Oct-4 as compared to a single knockdown of either HDAC7 or HDAC11.

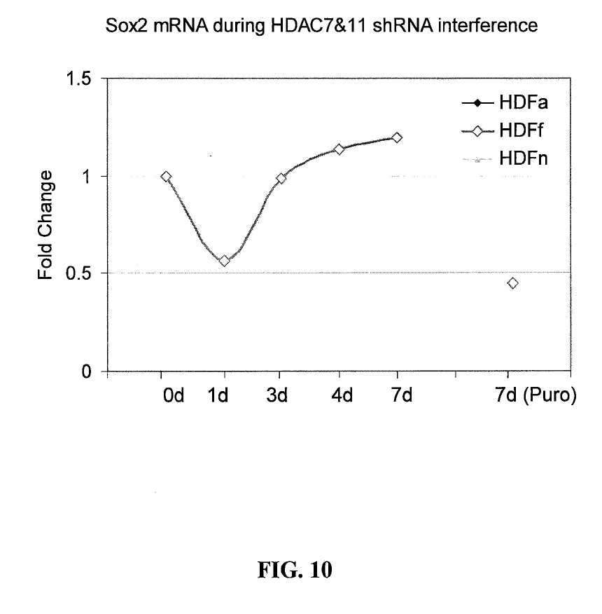

As reported in FIG. 10, Sox-2 expression occurred consistently in fetal human dermal fibroblasts. The expression of Sox-2 was maintained by double knockdown of HDAC7 and HDAC11.

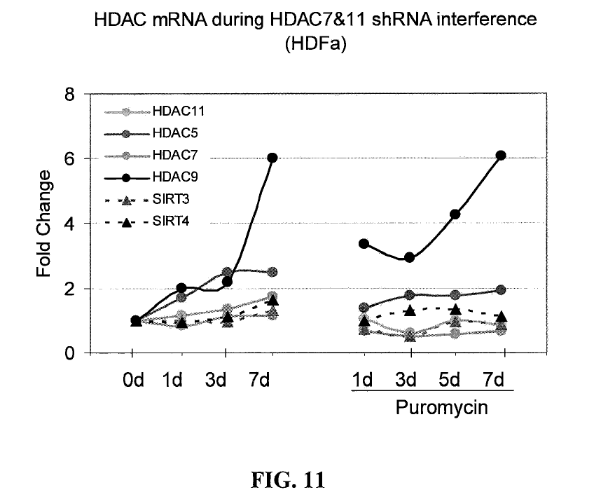

FIG. 11 reports the effects on the mRNA expression of various HDAC genes during dual HDAC7 and HDAC11 shRNA interference in adult human dermal fibroblasts. A robust increase in the expression of HDAC9 was observed. The expression of HDAC5 also was increased. Modest effects were observed on other genes (see FIG. 11).

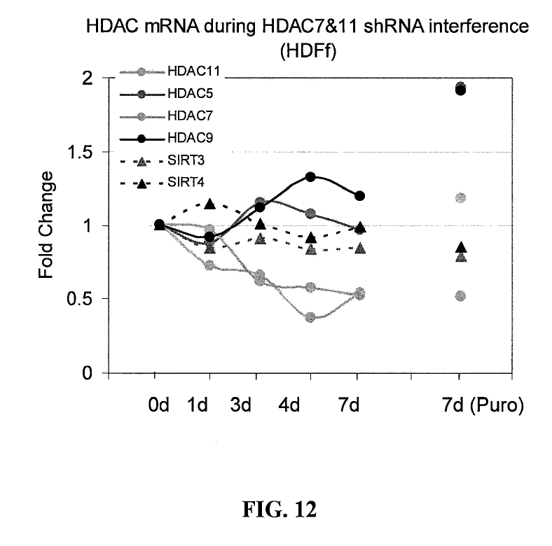

FIG. 12 reports the effects on the mRNA expression of various HDAC genes during dual HDAC7 and HDAC11 shRNA interference in fetal human dermal fibroblasts. A robust increase in the expression of HDAC9 was observed at day seven with puromyocin selection. The expression of various other HDAC genes was decreased at day seven with puromyocin selection (see FIG. 12).

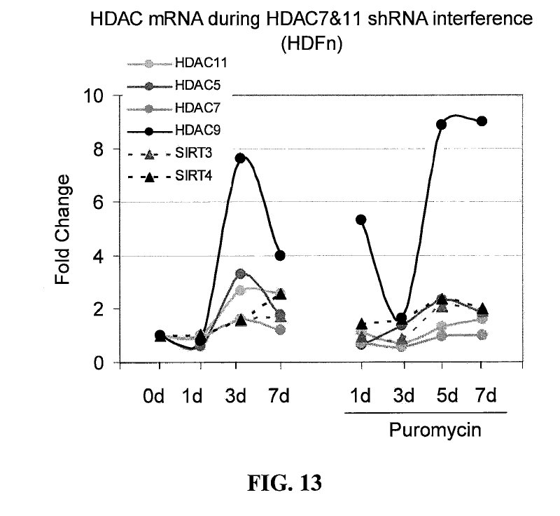

FIG. 13 reports the effects on the mRNA expression of various HDAC genes during dual HDAC7 and HDAC11 shRNA interference in neonatal human dermal fibroblasts. A robust increase in the expression of HDAC9 was observed at day without puromyocin selection and at day five with puromyocin selection. The expression of HDAC5 also was increased. Modest effects were observed on other genes (see FIG. 13).

These results demonstrate that a shRNA construct can be used to inhibit the expression of genes that code for an HDAC, and can induce expression of pluripotent genes, such as Oct-4 and Nanog, which are two genes involved in reprogramming a cell. Further, these results demonstrate that inhibition of HDACs can play an essential role in restoring differentiation potential to ac cell. The methods of the invention can be used to inhibit any HDAC or an HDAC related protein, either in structure or function.

To account for any compensatory pathway, redundant pathways, or compensatory and redundant pathways, more than one HDAC or any other protein involved in silencing of pluripotency genes may be inhibited. One or more proteins from the same family of inhibitory proteins, or two or more proteins from two different families of inhibitory proteins may be inhibited. One efficient mechanism for reprogramming a cell may to inhibit multiple proteins within the compensatory, redundant or compensatory and redundant pathways. Proteins that function within this inhibitory pathway may be inhibited by shRNA, HDAC inhibitors, small molecule inhibitors or any combination of the above-recited.

### Example 4

The effect of HDAC7 shRNA lentiviral infection on the expression of HDAC11, was tested. Three types of human dermal fibroblasts were used: adult human dermal fibroblasts (HDFa), neonatal human dermal fibroblasts (HDFn), and fetal human dermal fibroblasts (HDFf).

Methods

Cell culture. Human dermal fibroblasts were purchased from Cell Applications (San Diego, Calif.), and were maintained at 37° C. in 95% humidity and 5% CO₂ in Fibroblast growth medium (Cell Applications, San Diego, Calif.).

Lentiviral Infection. Human dermal fibroblasts were infected with a shRNA construct. The shRNA construct was obtained from Dharmacon. The shRNA construct directed toward HDAC7a had the following sequence:

SEQ ID NO. 1: GCTTTCAGGATAGTCGTGA

The human dermal fibroblasts were infected with the shRNA following the manufacturer's instructions. HDF were cultured with an without puromycin selection and hES culture conditions (mTeSR Medium, Stem Cell Technology, Vancouver, BC, Canada) on matrigel (BD Biosciences, San Jose Calif.).

Quantitative RT-PCR. Expression of HDAC7a and HDAC11 was determined by real-time RT-PCR. Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of HDAC7a and HDAC11 was normalized to glyceraldehyde 3-phosphate-dehydrogenase (GAPD).

Results

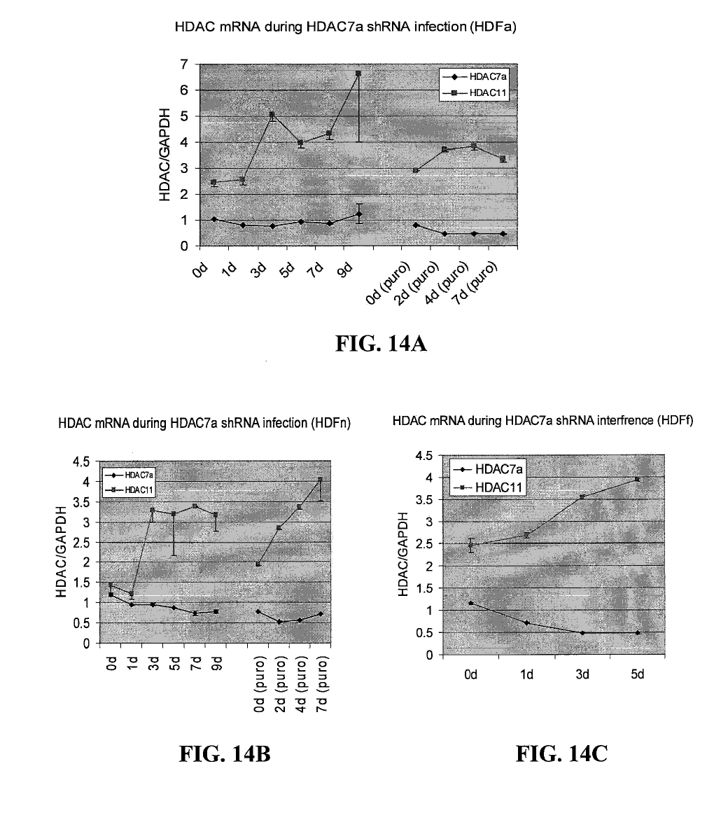

The expression of HDAC11 was increased, while the expression of HDAC7a was decreased, in fetal human dermal fibroblasts infected with HDAC7a shRNA (FIG. 14A). Similar results were obtained with neonatal human dermal fibroblasts (FIG. 14B) and fetal human dermal fibroblasts (FIG. 14C). The increase in expression was observed both in the presence and absence of puromycin. HDAC11 expression was up-regulated in a compensatory fashion in all three cell types tested. Inhibiting the expression of a gene that codes for a regulatory protein, which is involved in decreasing expression of a pluripotent gene, may lead to an increase in expression of other genes coding for a regulatory protein. Multiple agents targeted to a single family of regulatory proteins or multiple families of regulatory proteins may be an efficient means to reprogram a cell. The agents include but are not limited to small molecule inhibitors and shRNA constructs.

### Example 5

Cells infected with lentivirus shRNA directed to HDAC7, HDAC11 or DNMT1 were stained and visualized for expression of pluripotent genes. Protein expression of Oct-4 and Sox-2 was analyzed in this example, but one of ordinary skill in the art will understand the methods of the invention can be used to increase expression of any gene involved in reprogramming or restoring differentiation potential to a cell.

Methods

Cell culture. Fetal human dermal fibroblasts were purchased from Cell Applications (San Diego, Calif.), and were maintained at 37° C. in 95% humidity and 5% CO₂ in Fibroblast growth medium (Cell Applications, San Diego, Calif.).

Lentiviral Infection. Fetal human dermal fibroblasts were infected with one of the following compositions: (1) shRNA lentivirus directed to DNMT1; (2) shRNA lentivirus directed toward HDAC7; (3) shRNA lentivirus directed toward DNMT1 and HDAC7; and (4) shRNA lentivirus directed toward HDAC7a and HDAC11. The shRNA construct was obtained from Dharmacon. The shRNA construct directed toward HDAC7a had the following sequence:

SEQ ID NO. 1: GCTTTCAGGATAGTCGTGA

An shRNA construct with the following sequence was directed against HDAC11:

SEQ ID NO. 2: AGCGAGACTTCATGGACGA

In addition, an shRNA construct with the following sequence was directed against HDAC11:

SEQ ID. NO. 3: TGGTGGTATACAATGCAGG

The shRNA construct directed toward DNMT1 had the following sequence:

SEQ ID NO. 4: GTCTACCAGATCTTCGATA

The human dermal fibroblasts were infected with the shRNA following the manufacturer's instructions. HDF were cultured with an without puromycin selection and hES culture conditions (mTeSR Medium, Stem Cell Technology, Vancouver, BC, Canada) on matrigel (BD Biosciences, San Jose Calif.).

Immunohistochemistry. For immunohistochemistry, target shRNA-infected and control cells were grown on chambered slides (LabTek, Napersville, Ill.). Cells were then be fixed with 4% paraformaldehyde, and incubated with a specific antibody directed against pluripotency marker Oct3/4 (Abeam, Cambridge, Mass.) following the manufacturer's protocol. Staining of Oct3/4 was visualized as a red color. The nucleus was visualized with DAPI staining (Vectorshield), which appeared as a blue color.

Results

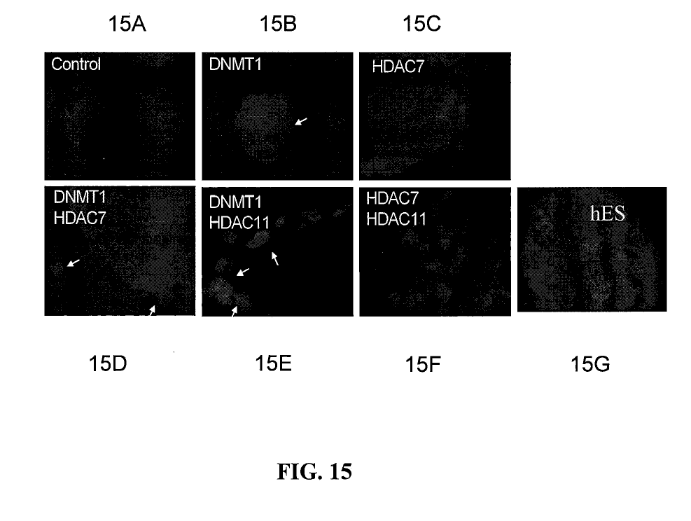

Oct-4 protein expression was increased in fetal human dermal fibroblasts (HDFf) by shRNA interference. FIG. 15A is a photograph of HDFf without infection (negative control). FIG. 15G is a photograph of human embryonic stem cells (positive control). In the negative control cells, little expression of Oct-4 protein was detected. FIG. 15B is a photograph of HDFf cells infected with shRNA directed toward DNMT1. Oct-4 protein expression is clearly increased when cells are exposed to DNMT1 shRNA. HDFf cells infected with HDAC7 shRNA show minimal detection of Oct-4 protein (FIG. 15C). This may be due to the processing of this particular sample.

Cells infected with DNMT1 and HDAC7 shRNA showed a dramatic increase in the expression of Oct-4 protein (FIG. 15D). The cells treated with both DNMT1 and HDAC7 shRNA produce an expression pattern very similar to human embryonic stem cells (Invitrogen, Carlsbad, Calif.) (FIG. 15E). These data corroborate data presented herein that an increase in Oct-4 gene expression leads to an increase in Oct-4 protein expression. DNMT and HDAC11 have distinct functions with regard to regulation of activation of transcription and chromatin remodeling. The inhibition of members from two separate regulatory groups resulted in a dramatic increase in the expression of Oct-4. Oct-4 protein expression was also increased in cells infected with DNMT1 and HDAC11 (FIG. 15E). Inhibition of DNMT1 and multiple HDACs resulted in increase in expression of Oct-4 protein.

There was no detectable increase in expression of Oct-4 in cells infected with HDAC7 and HDAC11 shRNA (FIG. 15F). This may be due a limitation of the experimental system. Alternatively, this result may suggest for optimal increase in expression of pluripotent genes, multiple pathways should be inhibited. Inhibiting the expression of genes that code for proteins that function in distinct regulatory complexes may result in higher expression levels of pluripotent genes. Any member of any regulatory complex may be inhibited.

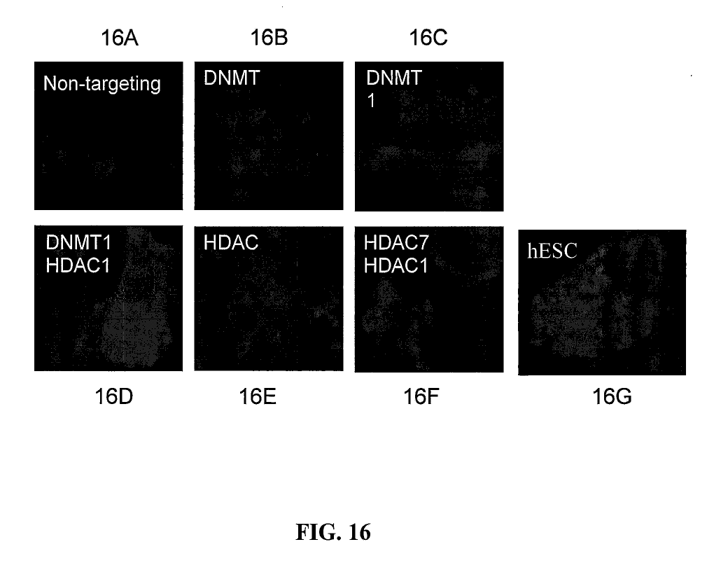

Sox-2 protein expression was increased in fetal human dermal fibroblasts (HDFf) by shRNA interference. FIG. 16A is a photograph of HDFf without infection (negative control). FIG. 16G is a photograph of human embryonic stem cells (FIG. 16G). In the negative control cells, little expression of Sox-2 protein was detected. FIG. 16B is a photograph of HDFf cells infected with shRNA directed toward DNMT1. Nuclear staining was visible, however only a modest amount of Sox-2 protein was detected. HDFf cells infected with HDAC7 and DNMT1 shRNA showed minimal detection of Sox-2protein (FIG. 16C). This may be due to the processing of this particular sample.

Cells infected with DNMT1 and HDAC11 shRNA showed a dramatic increase in the expression of Sox-2 protein (FIG. 16D). The inhibition of members from two separate regulatory groups resulted in a dramatic increase in the expression of Sox-2. Cells infected with HDAC7 shRNA showed minimal protein expression of Sox-2 (FIG. 16E). Sox-2 protein expression was also increased in cells infected with HDAC7 and HDAC11 (FIG. 16F). Inhibition of DNMT1 and multiple HDACs resulted in increase in expression of Sox-2 protein.

These results demonstrate that the inhibition of histone deacetylases and DNA methyl transferases increased the expression of pluripotent genes involved in reprogramming a cell. Two distinct shRNA constructs were targeted to two separate regulatory proteins, which resulted in a dramatic increase in expression of the Oct-4 and Sox-2 protein. Inhibiting more than one regulatory protein involved in inhibiting or repressing transcription of pluripotent genes may be an efficient mechanism to reprogram a cell and restore differentiation potential to a cell.

The inhibition of histone deacetylases and related family members can be used to increase the expression of pluripotent genes, and can be used to reprogram a differentiated cell. These reprogramming methods are independent of eggs, embryos or embryonic stem cells. Furthermore, these methods do not rely on viral vectors, which can have harmful effects. These methods are also independent of oncogenes, such as c-myc and Klf4.

In addition, the methods of the present invention can be used to reprogram a differentiated cell in the absence of somatic cell nuclear transfer (SCNT). SCNT is very inefficient and has posed a significant limitation on the field of reprogramming. The present methods alleviate the need for SCNT.

The present methods have demonstrated an increase in expression of the endogenous pluripotent genes and proteins, as opposed to measuring effects on an artificial vector with a strong reporter element. An artificial vector does not have the same chromatin structure as the endogenous gene, nor does it have other genes, and promoter elements to create the environment of the genome. An artificial vector does not have many of the natural elements needed to recapitulate the environment of the natural genome. The results presented herein represent effects obtained from treating human cells, and measuring the effects on the endogenous gene.

Finally, the data presented herein demonstrate that inhibiting or altering the function of histone deacetylases is one step involved in reprogramming a differentiated cell, and restoring differentiation potential.

### Example 6

A wide variety of small molecules, including small molecules known to inhibit histone deacetylases, were exposed to cells and analyzed for up-regulation of Oct-4. The following cell types were used: adult human dermal fibroblasts (HDFa), fetal human dermal fibroblasts (HDFf), and BJF cells.

Methods

Cell culture. Human dermal fibroblasts were purchased from Cell Applications (San Diego, Calif.), and were maintained at 37° C. in 95% humidity and 5% CO₂ in Fibroblast growth medium (Cell Applications, San Diego, Calif.).

Quantitative RT-PCR. Expression of Oct-4 was determined by real-time RT-PCR. Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of Cot-4 was normalized to glyceraldehyde 3-phosphate-dehydrogenase (GAPD).

Results

Table III provides the effects of small molecule inhibitors on Oct-4 gene expression in human somatic cells. mRNA levels were determined by real time qRT-PCR and normalized with GAPDH. The abbreviations are as follows: HDACi (histone deacetylase inhibitor DNMTi (DNA methyltransferase inhibitor); SIRTi (sirtuin, Class III HDAC inhibitor); LdeMi (lysine demethylase inhibitor); SIRT1a (sirtuin 1 activator); TSAi (trichostatin A inhibitor); HDMi/LSD1i (histone demethylase inhibitor/Lysine demethylase 1 inhibitor).

TABLE III
Small molecules and resulting effects on expression of Oct-4.

| Rx | Target action | Dose | | Rx Time | Cell Type | Oct4 FC | p < |
|---|---|---|---|---|---|---|---|
| VPA | Class I HDACi | 5 | mM | 5 | HLFa, f | 4(a), 5(f) | 0.01 |
| RG108 | DNMTi | 500 | uM | 5 | HLFa | <2 | 0.07 |
| Hydralazine HCL | DNMTi | 500 | uM | 2 | HDFa | <2 | 0.05 |
| ALA | HDACi | 50 | uM | 4 | HDFa | 3.5 | 0.0001 |
| Biotin | HDACi | 500 | uM | 4 | HDFa | <2 | 0.07 |
| Nicotinamide | SIRTi | .028 | mM | 4 | HDFa | 2.3 | 0.01 |
| Procaine HCL | DNMTi | 500 | uM | 8 | HDFa | <2 | 0.02 |
| Na Phenylbutyrate | HDACi | 2.5 | mM | 4 | HDFa | 2.5 | 0.06 |
| SIRT1 Activator 3 | SIRT1a | 500 | uM | 4 | HDFa | 2 | 0.01 |
| CAY 10433 | HDACi | 50 | uM | 4 | HDFa | <2 | 0.05 |
| Depudecin | HDACi | 50 | uM | 4 | HDFa | <2 | 0.01 |
| EX-527 | SIRTi | 50 | uM | 4 | HDFa | 2 | 0.01 |
| Splitomycin | HDACi | .5 | mM | 4 | HDFa | 2 | 0.02 |
| ITSA | TSAi | .05 | mM | 4 | HDFa | <2 | 0.03 |
| Valproxam | HDACi | .5 | mM | 4 | HDFa | <2 | 0.02 |
| Resveratrol | SIRT1a | .5 | mM | 4 | BJF | <2 | 0.01 |
| 2-PCPA | HDMi/LSD1i | 1 | mM | 8 | BJF | <2 | 0.001 |
| Tranylcypromine | HDMi/LSD1i | 2.5 | mM | 4 | HDFa | 2.7 | no stats |
| EGCG | DNMTi | 1 | uM | 4 | BJF | <2 | 0.09 |
| RO-31-8220 | SIRTi | 2.5 | mM | 4 | BJF | <2 | no stats |
| Sirtinol | HDA | .05 | mM | 7 | BJF | <2 | 0.006 |
| Psammaplin-A | DNA | 1.5 | mM | 7 | BJF | 2.2 | 0.000 |
| Zebularine | DNMTi | .05 | mM | 6 | BJF | <2 | 0.001 |

The compounds recited in Table III are meant to be a representative list of compounds, as opposed to an exhaustive list. One of ordinary skill in the art will understand that compounds with similar structure or similar function are encompassed with the methods, compositions and kits of the invention. Additional compounds within the same family of recited compounds also are included.

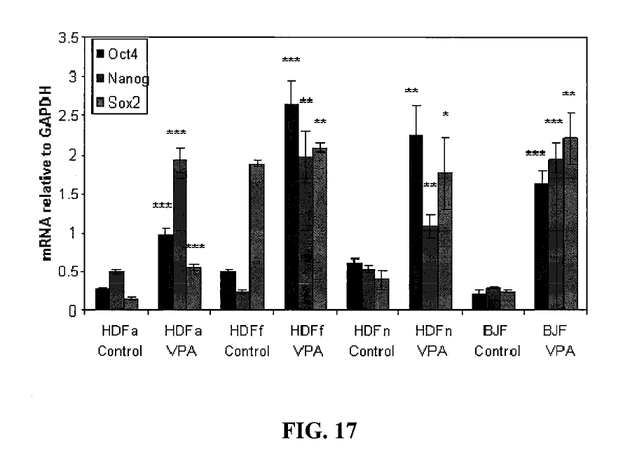

In addition to an increase in expression of Oct-4, exposing cells to VPA (5 mM) also caused an increase in expression of Sox-2 and Nanog (FIG. 17). Pluripotency gene expression was increased in multiple dermal fibroblast cell types following exposure to VPA for 4-6 days. mRNA levels were determined by real time qRT-PCR and normalized with GAPDH. Exposing a variety of different cell types to an HDAC inhibitor caused an increase in expression of pluripotency genes, thereby demonstrating the universal effect HDAC inhibitors and modulators can have on reprogramming.

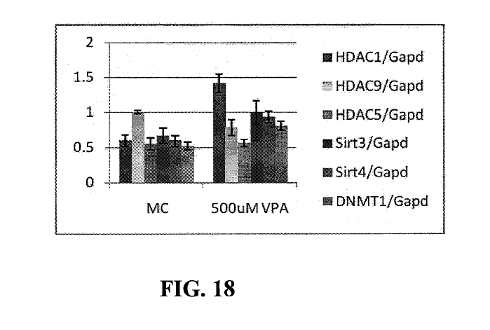

As was observed with shRNA constructs directed toward various HDACs, exposing cells to the HDAC inhibitor VPA led to an increase in gene expression of negative regulatory components such as HDAC molecules and DNA methyltransferases. FIG. 18 reports the effects of 500 μM VPA on HDAC and DNMT1 gene expression in BJFs. Cells were exposed to VPA for 7 days. HDAC1, SIRT4, and DNMT1 were significantly u-regulated compared to control cells. Statistical significance was as follows: HDAC1 (p<0.01); SIRT4 (p, 0.05) and DNMT1 (p<0.005).

Multiple targets involved in maintaining a specific cell type may need to be inhibited to achieve efficient reprogramming. Multiple regulatory pathways likely are involved in maintaining a particular cell type. A single agent or more than one agent may be used to inhibit regulatory pathways. Multiple agents may target the same protein, proteins within the same family, proteins with similar function, proteins with distinct function, different proteins, and proteins classified in distinct families. The agents can be similar in chemical structure and function or have distinct chemical structure and function.

Multiple agents can be administered simultaneously or sequentially. Agents can be administered at any time period from one another including 0.1-1 minute, 1-5 minutes, 5-20 minutes, 20-40 minutes, 40-60 minutes, 1-5 hours, 5-10 hours, 10-24 hours, 24-48 hours, 48-72 hours, 72-9 hours, 96-120 hours, 120-144 hours, 144-168 hours, and 168-500 hours.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference herein.

What is claimed is:

1. A method for reprogramming a cell comprising: exposing a population of cells to an agent that inhibits activity, expression, or activity and expression of a HDAC, wherein the inhibition of at least one HDAC leads to an increase in expression of other HDACs; exposing said population of cells to a second agent that inhibits activity, expression, or activity and expression of the other HDACs; selecting a cell that expresses a cell surface marker indicative of a pluripotent cell, and expanding said selected cell to produce a population of cells, wherein differentiation potential has been restored to said cell.

2. The method of claim 1, wherein said selecting a cell further comprises comparing phenotypes of the cell prior to and after exposure to said first and second agents, and identifying a cell with a phenotype consistent with a pluripotent cell.

3. The method of claim 1, wherein said selecting a cell further comprises using an antibody directed to protein coded for by a pluripotent gene or a cell-surface marker.

4. The method of claim 3, wherein said cell surface marker is selected from the group consisting of: SSEA3, SSEA4, Tra-1-60, and Tra-1-81.

5. The method of claim 1 further comprising: prior to expanding said cell, comparing chromatin structure of a pluripotent gene of said cell that exists prior to exposure to said first and second agents to the chromatin structure obtained after exposure to said agent.

6. The method of claim 5, wherein comparing chromatin structure comprises comparing acetylation state of histones.

7. The method of claim 5, wherein said pluripotent gene is selected from the group consisting of: Oct-4, Sox-2 and Nanog.

8. The method of claim 1, wherein said first or second agent is selected from the group consisting of: a small molecule inhibitor, a nucleic acid sequence, and a shRNA construct.

9. The method of claim 1, wherein said HDAC is selected from the group consisting of: HDAC1, HDAC2, HDAC3, HDAC8, HDAC4, HDAC5, HDAC6, HDAC7A, HDAC9, HDAC10, HDAC11, SIRT1, SIRT2, SIRT3, SIRT4, SIRT5, SIRT6, and SIRT7.

10. The method of claim 1, wherein said first agent is a small molecule inhibitor selected from the group consisting of: VPA, valproxam, sodium phenylbutyrate, biotin, and ALA.

11. The method of claim 1, wherein said population of cells are exposed to said first and second agent simultaneously.

12. A method for reprogramming a cell comprising: exposing a cell to a first agent that inhibits that activity, expression, or expression and activity of a HDAC; wherein the inhibition of at least one HDAC leads to an increase in expression of a second regulatory protein; exposing said cell to a second agent that inhibits the activity, expression or expression and activity of a second regulatory protein, wherein said second regulatory protein has a distinct function from the HDAC, and selecting a cell, wherein differentiation potential has been restored to said cell.

13. The method of claim 12, wherein said cell is exposed to said first and second agent simultaneously.

14. The method of claim 12, wherein selecting a cell comprises isolating a cell using an antibody directed to a protein coded for by a pluripotent gene or a cell-surface marker.

15. The method of claim 12, wherein said first and second agents are selected from the group consisting of: a small molecule inhibitor, a nucleic acid sequence, and a shRNA construct.

16. The method of claim 12, wherein said second regulatory protein is selected from the group consisting of: a histone acetyltransferase, a lysine methyltransferase, a histone methyltransferase, a histone demethylase, a lysine demethylase, a sirtuin, and a sirtuin activator.

17. An enriched population of reprogrammed cells produced according to a method comprising: exposing a population of cells to an agent that inhibits activity, expression, or activity and expression of a histone deacetylase, wherein said inhibition of at least one HDAC leads to an increase in expression of a second regulatory protein; exposing said cell to a second agent that inhibits the activity, expression or expression and activity of said second regulatory protein; and selecting a cell that express a cell surface marker indicative of a pluripotent cell.

18. The enriched population of reprogrammed cells of claim 17, wherein said first or second agent is selected from the group consisting of: a small molecule inhibitor, a nucleic acid sequence, and a shRNA construct.

19. The enriched population of reprogrammed cells of claim 17, wherein the reprogrammed cell expresses a cell surface marker selected from the group consisting of: SSEA3, SSEA4, Tra-1-60, and Tra-1-81.

20. The enriched population of reprogrammed cells of claim 17, wherein said reprogrammed cells account for at least 60% of the population.
